SEQUENCE LISTING (16 sequences)

SEQ ID NO: 1 - protein, 365 aa, Artificial Sequence
  Full-length H7-HA1 construct
MANKHLSLSLFLVLLGLSASLASGDKICLGHHAVSNGTKVNTLTERGVEVVNATETVERTNVPRICSKGKRTVDLGQCGLLGTITGPPQCDQFLEFSADLIIERREGSDVCYPGKFVNEEALRQILRESGGINKETMGFTYSGIRTNGATSACKRSGSSFYAEMKWLLSNTDNAAFPQMTKSYKNTRKDPALIIWGIHHSGSTTEQTKLYGSGSKLITVGSSNYQQSFVPSPGARPQVNGQSGRIDFHWLMLNPNDTVTFSFNGAFIAPDRASFLRGKSMGIQGGVQVDASCEGDCYHSGGTIISNLPFQNINSRAVGKCPRYVKQESLMLATGMKNVPELPKGRGLFLVPRGSSGHHHHHHDEL

SEQ ID NO: 2 - DNA, 1098 bp, Artificial Sequence
  Full-length H7-HA1 construct
atggcaaacaagcacctaagtctgagtctgttcctggtattgctgggactttcagcttctttggcaagtggtgataagatctgtctaggtcatcatgctgtaagtaacggaacaaaggtaaacacgctaacggagagaggagttgaagttgttaacgcaacagagacggttgaaagaaccaacgttccaagaatctgtagtaagggaaagaggaccgttgatctcggacaatgtggattgttgggaactatcacaggaccaccacaatgtgatcaattcctcgaattctccgctgatttgatcatcgaacgacgagaaggatcagatgtttgttaccctggtaagttcgttaacgaggaagcactcagacaaattctcagagaatccggtggtattaacaaggagaccatgggtttcacttactccggtattagaaccaatggtgcaacctcagcttgcaaacgtagtggttcatctttctacgcagaaatgaagtggttgctttcaaatactgacaatgccgcttttcctcagatgactaagtcctacaagaatacacggaaggaccctgctttgataatttggggtatacaccactccggatcaacaacagagcagactaaactttatggtagcggtagcaaacttattactgtcggtagcagcaattatcagcagagctttgtcccttctccgggagcaaggccacaagttaatggacagtctggtcgtattgattttcattggcttatgcttaatcctaatgacacagtcactttttcttttaatggcgcctttattgccccggacagggcctcttttcttaggggcaaatctatgggcattcaggggggggtgcaagtggatgcttcttgcgagggggattgctatcattctggcggcactataatttctaatttaccatttcaaaatataaattcacgggctgtggggaaatgccctaggtatgtgaaacaggagtcacttatgttagctacagggatgaaaaatgtgcccgagttacccaaaggccgtgggttatttttagttccgcggggttcatctgggcatcatcaccatcaccacgatgagttatga

SEQ ID NO: 3 - protein, 325 aa, Artificial Sequence
  Truncated H7-HA1 construct
MANKHLSLSLFLVLLGLSASLASGDKICLGHHAVSNGTKVNTLTERGVEVVNATETVERTNVPRICSKGKRTVDLGQCGLLGTITGPPQCDQFLEFSADLIIERREGSDVCYPGKFVNEEALRQILRESGGINKETMGFTYSGIRTNGATSACKRSGSSFYAEMKWLLSNTDNAAFPQMTKSYKNTRKDPALIIWGIHHSGSTTEQTKLYGSGSKLITVGSSNYQQSFVPSPGARPQVNGQSGRIDFHWLMLNPNDTVTFSFNGAFIAPDRASFLRGKSMGIQGGVQVDASCEGDCYHSGGTIISNLPLVPRGSSGHHHHHHDEL

SEQ ID NO: 4 - DNA, 978 bp, Artificial Sequence
  Truncated H7-HA1 construct
atggcaaacaagcacctaagtctgagtctgttcctggtattgctgggactttcagcttctttggcaagtggtgataagatctgtctaggtcatcatgctgtaagtaacggaacaaaggtaaacacgctaacggagagaggagttgaagttgttaacgcaacagagacggttgaaagaaccaacgttccaagaatctgtagtaagggaaagaggaccgttgatctcggacaatgtggattgttgggaactatcacaggaccaccacaatgtgatcaattcctcgaattctccgctgatttgatcatcgaacgacgagaaggatcagatgtttgttaccctggtaagttcgttaacgaggaagcactcagacaaattctcagagaatccggtggtattaacaaggagaccatgggtttcacttactccggtattagaaccaatggtgcaacctcagcttgcaaacgtagtggttcatctttctacgcagaaatgaagtggttgctttcaaatactgacaatgccgcttttcctcagatgactaagtcctacaagaatacacggaaggaccctgctttgataatttggggtatacaccactccggatcaacaacagagcagactaaactttatggtagcggtagcaaacttattactgtcggtagcagcaattatcagcagagctttgtcccttctccgggagcaaggccacaagttaatggacagtctggtcgtattgattttcattggcttatgcttaatcctaatgacacagtcactttttcttttaatggcgcctttattgccccggacagggcctcttttcttaggggcaaatctatgggcattcaggggggggtgcaagtggatgcttcttgcgagggggattgctatcattctggcggcactataatttctaatttaccattagttccgcggggttcatctgggcatcatcaccatcaccacgatgagttatga

SEQ ID NO: 5 - protein, 356 aa, Artificial Sequence
  Truncated H5-HA1 construct
MGWSWIFLFLLSGAAGVHCDQICIGYHANNSTEQVDTIMEKNVTVTHAQDILEKAHNGKLCSLNGVKPLILRDCSVAGWLLGNPMCDEFLNVPEWSYIVEKDNPVNGLCYPGDFNDYEELKHLLSSTNHFEKIQIIPRSSWSNHDASSGVSSACPYNGRSSFFRNVVWLIKKNSAYPTIKRSYNNTNQEDLLVLWGIHHPNDAAEQTKLYQNPTTYVSVGTSTLNQRSVPEIATRPKVNGQSGRMEFFWTILKPNDAINFESNGNFIAPEYAYKIVKKGDSAVMKSGLEYGNCNTKCQTPMGAINSSMPFHNIHPLTIGECPKYVKSDRLVLATGLRNVLVPRGSSGHHHHHHDEL

SEQ ID NO: 6 - DNA, 1070 bp, Artificial Sequence
  Truncated H5-HA1 construct
atgggatggagttggatattcctgttcctactatccggtgcagctggagtacattgtgatcaaatatgtatcggataccatgctaataactccactgaacaggtagatacgatcatggaaaagaacgtaacggtcactcatgcacaagatatcctggaaaaggcacataacggtaagctatgttctctgaacggtgttaagccacttatcttgagagattgttccgtagcaggttggcttcttggaaatcctatgtgtgatgaattcttaaacgtccctgaatggtcttacatcgttgaaaaggataaccctgttaacggactgtgttacccaggtgatttcaacgattacgaagaactcaagcatctcctctcttctacaaaccatttcgaaaagatacagattattccccggagttcctggtctaaccatgatgcaagtagcggagtttcaagtgcatgcccttataatggaagaagttctttctttcgaaacgtggtttggctcattaagaagaatagcgcttacccaacaattaagcgtagctacaataataccaatcaggaggacttgcttgtcttgtggggaattcaccacccaaatgacgctgctgaacaaactaagttgtatcagaatccgaccacttatgtgagcgttggtacatcaactttgaatcagagaagcgttcctgagattgctacacgaccaaaagttaatggtcaatcaggaagaatggagtttttttggaccattcttaagcccaatgacgccattaattttgagtcaaatgggaattttattgccccagagtatgcttataagatagtgaaaaaaggcgacagtgccgtgatgaaatcaggcttggagtatggtaattgcaataccaaatgccaaacacctatgggcgccattaattcttcaatgccgtttcacaatatacaccctttgactattggggagtgcccaaaatatgtcaaatctgaccgtcttgtgcttgctacagggcttaggaatgtgttagttccgcggggttcatctgggcatcatcaccatcaccacgatgagttatg

SEQ ID NO: 7 - protein, 325 aa, Artificial Sequence
  Truncated H6-HA1 construct
MGWSWIFLFLLSGAAGVHCDKICIGYHANNSTTQVDTILEKNVTVTHSIELLETQKEERFCKILNKAPLDLGECTIEGWILGNPQCDLLLGDQSWSYIVERPTARNGICYPGVLNEVEELKALIGSGEKVERFEMFPRNTWRGVDTNSGVTKACPSSTGGSSFYRNLLWIIKSKSAAYPVIKGTYNNTGNQPILYFWGVHHPPDTNEQNTLYGSGDRYVRMGTESMNFAKGPEIAARPAVNGQRGRIDYYWSVLKPGETLNVESNGNLIAPWYAYKFVSTSNKGAVFKSNLPVEDCHAICQTAAGVLRLVPRGSSGHHHHHHDEL

SEQ ID NO: 8 - DNA, 978 bp, Artificial Sequence
  Truncated H6-HA1 construct
atgggatggagctggatcttcctgttcctgctgagcggcgctgctggagtacactgtgataaaatctgcatcggataccacgctaacaatagcacaacccaggtggatacaatcctggagaagaacgtgacagtgactcactccatcgagctgctggaaactcagaaagaggaaagattttgcaagattctgaacaaggctccactggatctgggagagtgcacaatcgaaggatggattctgggaaatcctcagtgtgatctgctgctgggagatcagagctggagctacatcgtggagagacccaccgccagaaacggcatctgctaccccggcgtgctgaacgaggtggaggagctgaaggccctgatcggcagcggcgagaaggtggagagattcgagatgttccctaggaacacatggaggggcgtggacacaaacagcggcgtgacaaaggcctgccccagcagcaccggcggcagcagcttctacagaaacctgctgtggatcataaagagcaagagcgccgcctaccccgtgatcaagggcacctacaacaacaccggcaaccagcccatcctgtacttctggggcgtgcaccacccccccgacaccaacgagcagaacacactgtacggaagcggcgataggtacgtgagaatgggcactgaatccatgaattttgctaaaggacctgagatagctgctagacccgccgttaacggccagagaggcagaatcgattactactggagcgtgctgaagcccggcgagacactgaacgtggagtctaacggcaacctgatcgccccttggtacgcctacaagttcgtgagcaccagcaacaagggagccgtgttcaagagcaacctgcccgtggaggattgccacgccatctgccagaccgccgccggcgtgctgagactggtgcccaggggcagcagcggccaccaccaccaccatcatgatgaactgtga

SEQ ID NO: 9 - protein, 313 aa, Artificial Sequence
  Truncated H9-HA1 construct
MGWSWIFLFLLSGAAGVHCDKICVGHQSTNSTETVDTLTETNVPVTQAKELLHTEHNGMLCATNLGRPLILDTCTIEGLIYGNPSCDMLLGGREWSYIVERPSAVNGTCYPGNIENLEELRTLFSSSSSYQRIQLFPDTIWNVTYTGTSKSCSDSFYRNMRWLTQKNGLYPIQDAQYTNNRGKDILFVWGIHHPPTDTAQTNLYTRTDTTTSVTTENLDRTFKPLIGPRPLVNGLIGRINYYWSVLKPGQTLRVRSNGNLIAPWFGHILSGESHGRILRTDLSSGNCVVQCQTEKGLVPRGSSGHHHHHHDEL

SEQ ID NO: 10 - DNA, 960 bp, Artificial Sequence
  Truncated H9-HA1 construct
gacaccggtatgggctggagctggatctttctgttcctgctgagcggcgccgcaggagtgcactgcgataaaatctgcgtgggacaccagagcacaaacagcacagagacagtggatacactgacagagactaacgtgcctgtgacacaggccaaggaactgctgcacacagagcacaacggcatgctgtgcgcaacaaacctgggcagaccactgattcttgatacatgcacaattgaaggtctgatctacggaaaccctagctgcgatatgctgctgggaggaagggagtggtcatacattgtggaaagacctagcgccgtgaacggcacttgctaccctggaaacattgagaatctggaagaactgagaaccctgttcagcagcagctcttcttaccagagaatccagctgtttcctgatacaatttggaatgtgacatacacaggcacatctaagagctgcagcgatagcttttatagaaacatgaggtggctgacacagaaaaacggcttataccccatccaggacgctcagtatacaaataatagaggaaaggatattctgtttgtgtggggaatccatcaccccccaacagatactgcacagacaaatctgtacaccagaactgatactacaacatctgtgacaacagaaaatctggatagaacctttaaaccactgatcggaccaagacctctggtgaatggactgattggaagaattaattactactggtctgtgctgaaacctggccagacactgagagtgaggagcaacggaaacctgatcgccccctggtttggacacattctgtctggagagagccacggaagaatcctgagaacagatctgagcagcggcaactgcgtggtgcagtgtcagacagagaagggactggtgccaagaggaagcagcggccaccaccaccaccatcacgatgagctgtgactcgaggac

SEQ ID NO: 11 - DNA, 30 bp, Artificial Sequence
  pEAQ-HT forward primer
acttgttacgattctgctgactttcggcgg

SEQ ID NO: 12 - DNA, 28 bp, Artificial Sequence
  pHEAQ-HT reverse primer
cgacctgctaaacaggagctcacaaaga

SEQ ID NO: 13 - DNA, 20 bp, Artificial sequence
  FSC5 forward primer
ggttttcgaacttggagaaa

SEQ ID NO: 14 - DNA, 24 bp, Artificial Sequence
  FSC5 reverse primer
agaaaaccgctcaccaaacataga

SEQ ID NO: 15 - protein, 544 aa, Artificial Sequence
  CHIR-AB1-HRP conjugate
MGWSWIFLFLLSGAAGVHCHHHHHHGSLVPRGSQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSGGGGSQQLPQPSLSLHPSQGVSLGDTVTLRCHLPRMAAWVQLWLNGTLRFDKEKDKEQDAAEFSFAVTNLEDAGTYQCRYQVSEPLWTSNQSDPVELVLTGAKEWSHGNLLVAVLRGCAAVLVFSLGLYFVLDARSLWTRRDESAGVAPEMPESVQFQGPSRDSEDLTYAEMPAAIPCSQSPTSPTAPQSPVIYTTVSTDLPR

SEQ ID NO: 16 - DNA, 1653 bp, Artificial Sequence
  CHIR-AB1-HRP conjugate
aaaaccggtatgggatggagctggatcttcctgttcctgctgagcggcgcagctggagtgcactgccaccaccatcaccaccatggaagcctggtgcctagaggatctcagctgactccaacattttatgacaacagctgcccaaatgtgagcaacattgtgagagatactattgtgaacgaactgagaagtgatcctagaatagcagctagcattttgaggctgcactttcatgattgtttcgtgaacggatgcgacgctagcattctgctggataacacaacatcttttaggactgagaaagatgcatttggtaatgctaatagcgcaaggggatttcctgtgattgatagaatgaaagctgcagttgaaagcgcatgcccaagaacagtgtcctgcgctgatctgctgactattgcagctcagcagagcgtgaccctggccggcggcccaagctggagagtgcctctgggaagaagggatagcctgcaggccttcctggacctggccaacgctaacctgcccgcccccttcttcaccctgcctcagctgaaggactcctttagaaacgtgggcctgaacagaagcagcgacctggtggctctgagcggcgggcacacctttggcaagaaccagtgcagattcatcatggataggctgtacaacttcagcaacacaggcctgcccgaccccaccctgaacacaacctacctgcagacactgagaggactgtgccccctgaacggcaacctgagcgccctcgtggatttcgatctgagaacccccacaatctttgataacaagtactacgtgaacctggaggagcagaaaggcctgattcagagcgatcaggagctgttcagcagcccaaatgccacagacaccatccccctggtgaggagcttcgctaacagcacccagactttcttcaacgccttcgtggaggccatggacagaatgggaaacatcacccccctgaccggcacccagggccagatcagactgaactgccgggtggtgaactccaacagcggcggcggcggcagccagcagctgccacagccaagcctgagcctgcaccctagccagggagtgagcctgggagatacagtgactctgagatgccacctgccaagaatggctgcttgggtgcagctgtggctgaacgggaccctgagatttgataaggaaaaggataaagaacaggatgccgctgagttttcttttgctgtgacaaatctggaagatgctggcacataccagtgcagatatcaggtgtctgagccactgtggacatctaatcagagcgatccagtggagctggtgctgactggagcaaaagaatggtctcatggaaacctgctggtggcagtgctgagaggatgcgccgcagtgctggtgtttagcctgggactgtacttcgtgctggatgctagaagcctgtggaccaggagagatgagtctgctggcgtggccccagagatgcctgaaagcgtgcagtttcagggacctagcagagactctgaagatctgacatacgctgagatgcctgcagctatcccatgttctcagtctcctacatctcctactgctccacagagccccgtgatctacacaactgtgagcacagatctgcctagatgactcgagaaa

(19) United States
(12) Patent Application Publication
(10) Pub. No.: US 2021/0349092 A1
(43) Pub. Date: Nov. 11, 2021

(54) Plant Produced Avian Influenza Antigens and Their Uses in Diagnostic Assays and Devices

(71) Applicants: University of Pretoria, Pretoria (ZA); CSIR, Pretoria (ZA)

(72) Inventors: Celia Abolnik, Waterkloof Ridge (ZA); Martha Magaretha O'Kennedy, Pretoria (ZA)

(21) Appl. No.: 17/286,375

(22) PCT Filed: Oct. 18, 2019
(86) PCT No.: PCT/IB2019/058897

§ 371 Date: Apr. 16, 2021

(30) Foreign Application Priority Data
Oct. 19, 2018 (GB) 1817082.9

(51) Int. Cl. G01N 33/569; C07K 16/10; C12N 15/82; G01N 33/53; G01N 33/543

(52) U.S. Cl. CPC G01N 33/56983; C07K 16/1018; C12N 15/8202; G01N 2333/902; G01N 33/543; G01N 2333/916; G01N 33/5302

(57) ABSTRACT

The present invention relates to a method of detecting the presence of an antibody to an avian influenza hemagglutinin antigen in a sample from a subject, wherein the antibody binds to an epitope of an avian influenza hemagglutinin antigen, comprising the steps of cloning a nucleic acid encoding an avian influenza antigen into a vector, infiltrating a plant cell with the vector, recovering the antigen from the plant, contacting the sample with the antigen, and detecting the formation of an antibody-antigen complex, wherein the antibody-antigen complex comprises antibodies in the sample bound to the antigen and wherein the formation of an antibody-antigen complex confirms that the subject has been exposed to an avian influenza hemagglutinin antigen. The present invention also relates to a device for assaying the presence of an antibody to avian influenza hemagglutinin antigen in a sample from a subject.

### BACKGROUND OF THE INVENTION

The present invention relates to a method of detecting the presence of an antibody to an avian influenza hemagglutinin antigen in a sample from a subject, wherein the antibody binds to an epitope of the avian influenza hemagglutinin antigen, the method comprising the steps of cloning a nucleic acid encoding a truncated H5, H6, H7 or H9 antigen into a vector; infiltrating a plant cell with the vector so as to express the truncated H5, H6, H7 or H9 antigen in the plant cell; recovering the truncated H5, H6, H7 or H9 antigen expressed by the plant; contacting the sample with at least one of the truncated H5, H6, H7 or H9 antigens, wherein the antigens comprise SEQ ID NO:3, 5, 7 or 9; and detecting the formation of an antibody-antigen complex, wherein the antibody-antigen complex comprises antibodies in the sample bound to one or more of the truncated H5, H6, H7 or H9 antigens and wherein the formation of an antibody-antigen complex confirms that the subject has been exposed to an avian influenza hemagglutinin antigen. The present invention also relates to a device for assaying the presence of an antibody to avian influenza hemagglutinin antigen in a sample from a subject using a plant produced CHIR-AB1-HRP conjugate.

The surface of the Influenza A virus (IAV) membrane contains two viral-encoded glycoproteins, namely hemagglutinin (HA or H) and neuraminidase (NA or N) that are the major antigenic determinants and in combination form the H/N serotype. Sixteen H and nine N glycoprotein types have been discovered in birds, and these may theoretically occur in any combination. Avian influenza (AI), especially when caused by the highly pathogenic (HPAI) H5 or H7 viral serotypes, is a serious disease of poultry with zoonotic potential (OIE, 2008). Three epidemiologically unrelated outbreaks of H5N2 HPAI in 2004, 2006, and 2011 had devastating economic impacts on the South African ostrich industry through control measures, restrictions on meat exports, and other socioeconomic factors (Moore et al., 2014). South Africa is the largest global producer of ostriches, and farming operations are concentrated in the Klein Karoo, a semi-desert region that spans the Eastern and Western Cape provinces. Ostriches are valued for their lean meat and their skins, which are used to produce luxury leather goods. Farmed ostriches are classified as poultry by the World Organisation for Animal Health (OIE); all guidelines for poultry surveillance and diagnosis, and regulations for control of AI, therefore apply (OIE, 2008). The extensive nature of ostrich farming production systems bears the continual risk of point introductions of avian influenza virus (IAV) from wild birds, the natural reservoirs (Webster et al., 1992). Prior to 2017, infection of commercial chickens in South Africa with IAV was limited to an endemic H6N2 infection (Rauff et al, 2016). In July 2017, the pandemic strain of HPAI H5N8 clade 2.3.4.4 reached South Africa via migratory birds. A devastating epidemic ensued, with commercial chickens, commercial ostriches, back yard and hobby birds as well as wild birds affected. Only the Limpopo and Northern Cape Provinces remained unaffected. Over five million chickens were culled and 70% of the layer hen population of the Western Cape Province was destroyed causing a 20% increase in national egg prices due to shortages (Abolnik et al., 2018). Epidemics of H9N2 influenza viruses causes severe disease and production problems in countries of the Middle East, Asia and more recently central Africa, but has not spread to South African poultry. Increasing detections of human infections with H9N2 poultry viruses have raised concerns that H9N2 could be a future pandemic strain (Rahimirad et al, 2016).

Serological testing of poultry for surveillance and to prove freedom of disease for international trade purposes is compulsory in South Africa. All poultry in South Africa (chickens and ostriches) are screened for the presence of H5, H6, H7 or H9 influenza on a bi-annual basis. Commercial ostriches are also tested pre-movement and pre-slaughter. The hemagglutination inhibition (HI) test is the OIE-recommended “golden standard” method for identifying serotype-specific antibodies in the sera of poultry. In South Africa, poultry serum is first screened by commercial ELISA tests that detect antibodies raised against a highly conserved protein of the influenza A virus group, for example matrix or nucleoprotein. Any positive reactors are tested further by HI assays, to determine the viral serotype to which the flock was exposed. Each positive sample is tested for the presence of anti-H5, -H6 and -H7 specific antibodies, using two viral antigens each with heterologous N types. Cross-reactions due to N-type antibodies and lack of a suitable test antigen panel causes serious complications in the interpretation of HI test results, often leading to the farm incorrectly being placed under quarantine. Furthermore, it was previously established that the HI test is less sensitive and specific when used with ostrich sera, missing up to 35% of H5 positive reactions (Abolnik et al 2013). Ostrich sera, as with all other non-gallinaceous sera, must be pre-absorbed with chicken red blood cells used in the HI assay to eliminate non-specific reactions in accordance with OIE guidelines (OIE, 2008).

The use of commercial AIV antibody ELISAs in South Africa since 2011 for AIV-group exposure screening has significantly improved the early detection and control of avian influenza infections. Similarly, serotype-specific ELISAs potentially offer significant benefits over HI tests, especially for ostriches and other non-gallinaceous species. ELISAs are automatable, not prone to subjective interpretation, require less test sera with no pre-treatment, completely eliminate N cross-reactions and are possibly more sensitive. Few H5 and H7 ELISA assays are available commercially (no H6 assay is commercially available), but the kits are produced abroad and, are expensive compared to HI and subject to exchange fluctuations.

Previously, an indirect H5 ELISA for ostriches was developed by producing a horseradish peroxidase (HRP) conjugate in chickens against ostrich IgY. The coating antigen, a poly histidine-tagged recombinant H5-HA1 protein (rH5-HA1) was expressed in humans cells by service provider Creative Diagnostics, New York, USA. The cost of the glycoprotein was the limiting factor in perusing a commercial product, and neither H6 nor H7 ELISAs explored (Abolnik et al., 2013).

Plant expression of viral glycoproteins has several advantages over traditional methods of preparing influenza antigens used in vaccines and diagnostic assays. The correct folding and glycosylation of the HA is essential to maintain biological functions such as immunogenicity and receptor-binding activity. Traditionally, IAVs are grown in embryonated Specific Pathogen Free (SPF) chicken eggs. High bio-containment facilities, typically BSL3, are required for the propagation of the live virus. Mammalian and insect cell culture systems similarly require sterile environments and highly-skilled technicians, and live genetically modified viruses are also required to be contained in a BSL3 environment. Prokaryotic systems, such as E. coli, are unable to express and glycosylate HA correctly, leading to insoluble protein aggregates, as well as cross-reactions with antibodies in chickens that have natural exposure to E. coli through their environment.

Biopharmaceutical proteins and vaccines are traditionally produced in bacteria, eggs, yeast and animal cell cultures and are well established industries. More recently, these molecules are being produced in plants, a method known as biopharming. Plant-based production systems have the substantial cost reduction, facile scalability and offer a low risk of contamination by endotoxins or human pathogens. In addition, plants are capable of introducing eukaryotic post-translational modifications such as glycosylation. These advantages however are molecule/product-specific and depend on the relative cost-efficiency of alternative sources of the same product (biosimilars) or improved products (biobetters). Plants are grown in enclosed greenhouse or growth room facilities, with highly regulated downstream processes to ensure product quality.

### SUMMARY OF THE INVENTION

The present invention relates to method of detecting the presence of an antibody to an avian influenza hemagglutinin antigen in a sample from a subject and to a device for assaying the presence of an antibody to avian influenza hemagglutinin antigen in a sample from a subject.

In a first aspect of the invention there is provided for a method of detecting the presence of an antibody to an avian influenza hemagglutinin antigen in a sample from a subject, wherein the antibody binds to an epitope of the avian influenza hemagglutinin antigen, the method comprising the steps of:

(i) cloning a codon optimised nucleic acid encoding a truncated H5, H6, H7 or H9 antigen into a vector; (ii) infiltrating a plant cell with the vector so as to express the truncated H5, H6, H7 or H9 antigen in the plant; (iii) recovering the truncated H5, H6, H7 or H9 antigen expressed by the plant; (iv) contacting the sample with at least one of the truncated H5, H6, H7 or H9 antigens, and (v) detecting the formation of an antibody-antigen complex, wherein the antibody-antigen complex comprises antibodies in the sample bound to one or more of the truncated H5, H6, H7 or H9 antigens;

wherein the formation of an antibody-antigen complex confirms that the subject has been exposed to an avian influenza hemagglutinin antigen.

In a first embodiment of this aspect of the invention the truncated H5, H6, H7 or H9 antigens comprise a sequence of SEQ ID NO:3, 5, 7 or 9, respectively. Those of skill in the art will appreciate that derivatives and variants of these sequences may have the same antigenic effect.

In a second embodiment of the invention the formation of the antibody-antigen complex is detected by either (i) the binding of a labelled secondary antibody to the antibody-antigen complex, or (ii) by the binding of a labelled secondary antigen to the antibody-antigen complex. It will be appreciated that the labelled secondary antibody may either be conjugated to or may be a genetic fusion with an indicator molecule.

In a third embodiment of the invention the labelled secondary antibody comprises the sequence of SEQ ID NO:15. Preferably, the labelled secondary antibody is the non-mammalian Fc-gamma receptor, CHIR-AB1, a member of the leukocyte receptor complex, that binds IgY with high affinity with its single Ig domain.

In a fourth embodiment of the invention the labelled secondary antigen is a truncated H5, H6, H7 or H9 antigen which is either conjugated to or is a genetic fusion with an indicator molecule.

In a preferred embodiment of the invention the indicator molecule is selected from horseradish peroxidase or alkaline phosphatase and the indicator molecule is a genetic fusion with the antigenic protein or a genetic fusion with the secondary antibody.

In a sixth embodiment of the invention the subject has been exposed to avian influenza.

It will be appreciated that the sample may be selected from the group consisting of blood, serum, plasma, saliva, conjunctival fluid urine and feces or any other bodily fluid.

In a seventh embodiment of the invention the at least one truncated H5, H6, H7 or H9 antigen includes an affinity tag, and wherein the affinity tag facilitates the purification of the antigen. In a preferred embodiment the affinity tag is a 6×-His tag.

In a further embodiment of the invention the at least one truncated H5, H6, H7 or H9 antigen is attached to or immobilized to a solid support. Preferably the solid support is selected from the group consisting of a bead, a flow path in a lateral flow immunoassay device, a well in a microtiter plate, or a flow path in a rotor. Those of skill in the art will appreciate that the formation of the antibody-antigen complex may be detected by one or more of the following: dip stick immunotesting, ELISA, flow cytometry, fluorescence, immunochip assay, immunochromatographic assay, immunoblot, latex agglutination, lateral flow assay, polarization, radioimmunoassay, and bead-based technology.

In a second aspect of the invention there is provided for a device for assaying for the presence of an antibody to avian influenza hemagglutinin antigen in a sample from a subject, comprising:

(i) at least one antigen comprising a sequence selected from SEQ ID NO:3, 5, 7 or 9; and (ii) a means for detecting the formation of an antibody-antigen complex between an antibody in the sample and the at least one antigen;

wherein the means for detecting the formation of an antibody-antigen complex comprises:

(a) a labelled secondary antibody; or (b) a labelled secondary antigen;

wherein binding of the labelled secondary antibody or labelled secondary antigen to the antibody-antigen complex confirms that the subject has been exposed to an avian influenza hemagglutinin antigen.

In a first embodiment of this aspect of the invention the labelled secondary antibody is either conjugated to or is a genetic fusion with an indicator molecule.

In a second embodiment of this aspect of the invention the labelled secondary antibody comprises the sequence of SEQ ID NO:15. Preferably, the labelled secondary antibody is the non-mammalian Fc-gamma receptor, CHIR-AB1, a member of the leukocyte receptor complex, that binds IgY with high affinity with its single Ig domain.

In a third embodiment of the invention the labelled secondary antigen is a truncated H5, H6, H7 or H9 antigen which is either conjugated to or is a genetic fusion with an indicator molecule.

In a preferred embodiment of the invention the indicator molecule is selected from horseradish peroxidase or alkaline phosphatase and the indicator molecule is a genetic fusion with the antigenic protein or a genetic fusion with the secondary antibody.

In a fifth embodiment of the invention the subject has been exposed to avian influenza. It will be appreciated that the sample may be selected from the group consisting of blood, serum, plasma, saliva, conjunctival fluid urine and feces or any other bodily fluid.

In a further embodiment of the invention the at least one truncated H5, H6, H7 or H9 antigen is attached to or immobilized to a solid support. Preferably the solid support is selected from the group consisting of a bead, a flow path in a lateral flow immunoassay device, a well in a microtiter plate, or a flow path in a rotor. Those of skill in the art will appreciate that the formation of the antibody-antigen complex may be detected by one or more of the following: dip stick immunotesting, ELISA, flow cytometry, fluorescence, immunochip assay, immunochromatographic assay, immunoblot, latex agglutination, lateral flow assay, polarization, radioimmunoassay, and bead-based technology.

### BRIEF DESCRIPTION OF THE FIGURES

Non-limiting embodiments of the invention will now be described by way of example only and with reference to the following figures:

FIG. 1: Possible ELISA formats

FIG. 2: pEAQ-HT with the rH5-HA1 insert (SEQ ID NO:6) at the Age I/Xho I restriction sites.

FIG. 3: pEAQ-HT with the rH7-HA1 insert (SEQ ID NO:4) at the Age I/Xho I restriction sites.

FIG. 4: Protein design for the recombinant rH7-HA1 protein (SEQ ID NO:1) [Influenza A virus (A/ostrich/South Africa/KVL/2012(H7N1)), protein ID ALJ33344.1. The plant Barley amylase signal peptide was selected and underlined in the sequence below, thrombin cleavage site typed in bold, C-terminal His-tag in italics and the C-terminal HDEL retention signal sequence is also shown.

FIG. 5: Protein design for truncated hemagglutinin rH7-HA1 protein (SEQ ID NO:3). The plant Barley amylase signal peptide was selected and is underlined, thrombin cleavage site shown in bold, C-terminal His-tag in italics and the C-terminal HDEL retention signal sequence is shown. The C-terminal 40 amino acids of the HA1 gene were removed and led to significant improvements in the expression of the protein, with no effect on antibody recognition.

FIG. 6: Protein design for a truncated version of the hemagglutinin rH5-HA1 protein (SEQ ID NO:5), [partial influenza A virus (A/ostrich/South Africa/C42 KF/2012(H5N2))] protein ID ALJ33273.1. The cleavage peptide PQRRKKRGLF of rH5-HA1 was removed. The murine signal peptide is underlined, thrombin cleavage site in bold, C-terminal His-tag are in italics and C-terminal HDEL retention signal sequence.

FIG. 7: Immunoblot detection of HA1 of both H5 and H7 subtypes produced in N. benthamiana. The Agrobacterium OD₆₀₀ of infiltration was ˜0.6. Lane 1, Biorad precision plus protein WesternC standard; lane 2-3, crude plant extract of rH5-HA1 in Bicine buffer; lanes 4-5, crude plant extract of rH5-HA1 in Tris-HCl buffer; lane 6, empty; lanes 7-8, crude plant extract of rH7-HA1 in Bicine buffer; lane 9, crude plant extract of rH7-HA1 in Tris-HCl buffer. Precision Plus Protein™ WesternC™ standards detected by Strep Tactin HRP-conjugate. Arrows indicates monomers (˜37 kDa and ˜44 kDa) and dimers (74 and 88 kDa) of rH7-HA1 and rH5-HA1, respectively.

FIG. 8: Immunoblot detection of HIS-tagged protein purification of rH5-HA1 and rH7-HA1 produced in N. benthamiana and probed with anti-HIS-HRP. The Agrobacterium OD₆₀₀ of infiltration was ˜0.6. Lane 1, Biorad precision plus protein WesternC standard; lane 2, pEAQ-HT empty vector; lanes 3-4, rH5-HA1 Bicine buffer extract, Ni-TED elute fraction elute1, lane 5, rH5-HA1 Bicine buffer extract, elute 2; lanes 6-7, rH5-HA1 Tris-HCl buffer extract, elute 1; lane 8, rH5-HA1 Tris-HCl buffer extract, elute 2; lanes 9-10, rH7-HA1 Bicine buffer extract, elute1; lane 11, rH7-HA1 Bicine buffer extract, elute 2; lanes 12-13, rH7-HA1 Tris-HCl buffer extract, elute 1; lane 14, rH7-HA1 Tris-HCl buffer extract, elute 2. Precision Plus Protein™ WesternC™ standards detected by Strep Tactin HRP-conjugate. Arrows indicates monomers (˜37 kDa and ˜44 kDa) and dimers (74 and 88 kDa) of rH7-HA1 and rH5-HA1, respectively.

FIG. 9: Immunoblot detection of rH5-HA1 (lanes 2 and 3) and rH7-HA1 (lanes 4 and 5) using H5N2 and H7N7-specific antisera raised in chickens sera, respectively.

FIG. 10: Immunoblot detection of mass purification of rH7-HA1 produced in N. benthamiana. The Agrobacterium OD₆₀₀ of infiltration was ˜0.6. Lane 1, Biorad precision plus protein WesternC standard; lane 2, pEAQ-HT empty vector negative control; lane 3, pEAQ-HT-gfp negative control; lane 4, crude plant extract of rH7-HA1 in Bicine buffer; lane 5, AKTA purified and concentrated; lanes 6-11, purified dialysed protein loaded 2, 4, 6, 10, 13 and 16 μl. Both monomers (˜37 kDa) and dimers (74 kDa) were detected of rH7-HA1. Precision Plus Protein™ WesternC™ standards detected by Strep Tactin HRP-conjugate.

FIG. 11: ELISA evaluating the appropriate dilution factor of H5 antigen, antiserum and conjugate. Specific pathogen free (SPF) chicken serum served as negative control antibodies whereas chicken H5N2 primary (1:1000 or 1:2000) served as primary antibody. Anti-chicken-HRP (1:5000 or 1:8000) was used as secondary antibody. Water vacuum infiltrated and un-infiltrated leaf extracts at 1:50, 1:100 and 1:200 served as negative controls and was probed with H5N2 (1:1000) and anti-chicken-HRP (1:5000). A working concentration of H5 antigen of 1:20 (0.15 μg per well) was considered optimal, and a cut-off point of OD₄₅₀=0.3.

FIG. 12: Immunoblot detection of HIS-tagged protein purification of rH5-HA1 produced in N. benthamiana and probed with anti-HIS-HRP. The Agrobacterium OD₆₀₀ was 2. MW, Biorad precision plus protein WesternC standard; lane 1, positive control, 72 ng; lane 2, pEAQ-HT empty vector; lanes 3, 7 and 11, crude extract; lanes 4-6, LBA4404 mediated rH5-HA1 protein production, 2, 10 and 16 μl, respectively; lanes 8-10, GV3101::pMP90 mediated rH5-HA1 protein production, 2, 10 and 16 μl, respectively; lanes 12-14, AGL-1 mediated rH5-HA1 protein production, 2, 10 and 16 μl, respectively. Precision Plus Protein™ WesternC™ standards detected by Strep Tactin HRP-conjugate. Arrows indicates monomers (˜44 kDa) and dimers (88 kDa) of rH5-HA1.

FIG. 13: Immunoblot detection of HIS-tagged protein purification of rH7-HA1 produced in N. benthamiana and probed with anti-HIS-HRP. The Agrobacterium OD₆₀₀ was 2. MW, Biorad precision plus protein WesternC standard; lane 1, positive control, 72 ng; lane 2, pEAQ-HT empty vector; lanes 3, 7 and 11, crude extract; lanes 4-6, LBA4404 mediated rH7-HA1 protein production, 2, 10 and 16 μl, respectively; lanes 8-10, GV3101::pMP90 mediated rH7-HA1 protein production, 2, 10 and 16 μl, respectively; lanes 12-14, AGL-1 mediated rH7-HA1 protein production, 2, 10 and 16 μl, respectively. Precision Plus Protein™ WesternC™ standards detected by Strep Tactin HRP-conjugate. Arrows indicates monomers (˜37 kDa) and dimers (74 kDa) of rH7-HA1.

FIG. 14: Protein design for a truncated version of the hemagglutinin rH9-HA1 protein (SEQ ID NO:9), [H9 hemagglutinin [Influenza A virus (A/chicken/Uganda/MUWRP-200192/2017(H9N2))] protein ID AVK87182.1. The murine signal peptide is underlined, thrombin cleavage site in bold, C-terminal His-tag are in italics and C-terminal HDEL retention signal sequence are indicated.

FIG. 15: pEAQ-HT with the rH9-HA1 insert (SEQ ID NO:10) at the Age I/Xho I restriction sites.

FIG. 16: Immunoblot detection of HIS-tagged protein purification of rH9-HA1 produced in N. benthamiana and probed with anti-HIS-HRP. Proteins were extracted five days post infiltration in a Bicine buffer. The Agrobacterium inoculum OD₆₀₀ was 2. MW, Biorad precision plus protein WesternC standard; lane 1, flow through of Ni-TED column; lanes 2-3, Agrobacterium strain AGL-1 mediated rH9-HA1 production, 4 and 8 μl (254 and 508 ng protein, respectively); lanes 4-5, Agrobacterium strain GV3101::pM90 mediated production of rH9-HA1, 4 and 8 μl (343 and 686 ng protein, respectively) and lanes 6-7, Agrobacterium strain LBA4404 mediated production of rH9-HA1, 4 and 8 μl (366 and 732 ng protein, respectively). Precision Plus Protein™ WesternC™ standards detected by Strep Tactin HRP-conjugate. Arrows indicate monomers (˜33 kDa) and dimers (66 kDa) of rH9-HA1.

FIG. 17: Immunoblot detection using H9 anti-serum targeting the rH9 HA1 antigen produced in N. benthamiana. Proteins were extracted five days post infiltration in a Bicine buffer. The Agrobacterium inoculum OD₆₀₀ was 2. MW, Biorad precision plus protein WesternC standard; lane 1, uninfiltrated leaf tissue; lanes 2-5, Agrobacterium strain LBA4404 mediated production of H9, loading 1, 2, 5 and 10 μl protein extract; lanes 6-9, Agrobacterium strain GV3101::pM90 mediated production of H9, loading 1, 2, 5 and 10 μl protein extract; lanes 10-13, Agrobacterium strain AGL-1 mediated production of H9, loading 1, 2, 5 and 10 μl protein extract. Arrows indicate monomers (˜33 kDa) and dimers (66 kDa) of rH9 HA1 and a partially denatured product of ˜48 kDa.

FIG. 18: Bis-Tris Bolt™ 4-12% SDS-PAGE of partially purified rH9 antigen. MW, SeeBlue® Plus2 Pre-Stained Standard. Lane 1, rH9 HA1 antigen produced in N. benthamiana. Proteins were extracted five days post infiltration in a Bicine buffer. The Agrobacterium inoculum OD₆₀₀ was 2. Arrows indicate monomers (˜33 kDa, fragment 1) and dimers (66 kDa, fragment 3) of rH9 HA1 and a partially denatured product of ˜48 kDa (fragment 2).

FIG. 19: Protein sequence coverage for recombinant H9 fragments (SEQ ID NO:9) (SDS PAGE, FIG. 18, fragments 1-3) that were analysed. LC-MS/MS based peptide sequence analysis for excised bands are as indicated. Peptides with >95% confidence are in bold text, <50% confidence underlined and no peptides identified for the regions of the sequences in italics text.

FIG. 20: Protein design for a truncated version of the hemagglutinin rH6-HA1 protein (SEQ ID NO:7), [H6 hemagglutinin [Influenza A virus (A/chicken/South Africa/BKR4/2012(H6N2))] protein ID ANZ03749.1. The murine signal peptide is underlined, thrombin cleavage site in bold, C-terminal His-tag are in italics and C-terminal HDEL retention signal sequence are indicated.

FIG. 21: pEAQ-HT with the rH6-HA1 insert (SEQ ID NO:8) at the Age I/Xho I restriction sites.

FIG. 22: Immunoblot detection of HIS-tagged protein purification of rH6-HA1 produced in N. benthamiana and probed with anti-HIS-HRP. Proteins were extracted five days post infiltration in a Bicine buffer (blots A-B) or Tris buffer (blots C-D). The Agrobacterium inoculum OD₆₀₀ was 2. MW, Biorad precision plus protein WesternC standard; lane 1, positive control (not visible); lane 2, crude; lanes 3 and 9, crude extract; lanes 4 and 10, Flow through; lanes 5 and 11, elute; lanes 6-8, concentrated H6 protein 2, 8 and 16 μl (blots A-D); lanes 12-14, concentrated H6 protein 2, 8 and 16 μl (blots A and C). Agrobacterium strain AGL-1 (blots A and C), LBA4404 (blots A and C) and GV3101::pM90 (blots B and D) mediated production of H6. Precision Plus Protein™ WesternC™ standards detected by Strep Tactin HRP-conjugate. Arrows indicate monomers (˜36 kDa) and dimers (72 kDa) of rH6-HA1.

FIG. 23: Immunoblot detection of rH6-HA1 (lanes 2 and 3) H6-specific antisera raised in chickens followed by anti-chicken-HRP secondary antibody. N. benthamiana plants were infiltrated with an Agrobacterium inoculum OD₆₀₀ of 2. MW, Biorad precision plus protein WesternC standard; lane 1, positive control, full length HA0 (62 kDa); lane 2, empty lane; lane 3-4 and 8-9, AGL-1 mediated production of H6, 8 and 16 μl respectively; lanes 5-6 and 10-11, GV3101::pM90 mediated production of rH6, 8 and 16 μl respectively. Leaf material was extracted using either a Bicine (lanes 3-6) or Tris-based (lanes 8-11) buffer. Precision Plus Protein™ WesternC™ standards detected by Strep Tactin HRP-conjugate. Arrows indicate monomers (˜36 kDa) and dimers (72 kDa) of rH6-HA1.

FIG. 24: Immunoblot detection of HIS-tagged protein purification of rH6-HA1 mass produced in N. benthamiana, extracted in a Bicine buffer (pH 8.4), purified, concentrated and dialysed in a phosphate buffer (pH 7.4) and finally probed with anti-HIS-HRP. The AGL-1 Agrobacterium OD₆₀₀ of infiltration was 2. MW, Biorad precision plus protein WesternC standard; lane 1, positive control; lanes 2-3, crude extract, 8 and 16 μl respectively; lane 4, truncated rH6-HA1 Ni-TED elute (16 μl); lanes 5-10, concentrated and dialysed protein 4, 6, 8, 10 and 16 μl, respectively. Arrows indicate monomers (˜36 kDa) and dimers (72 kDa). Precision Plus Protein™ WesternC™ standards detected by Strep Tactin HRP-conjugate.

FIG. 25: Bis-Tris Bolt™ 4-12% SDS-PAGE of partially purified rH6 HA1 antigen. MW, SeeBlue® Plus2 Pre-Stained Standard. Lanes 1-2, rH6 HA1 antigen produced in N. benthamiana. Proteins were extracted five days post infiltration in a Bicine buffer (lane 1) and Tris-HCl buffer (lane 2). The AGL-1 Agrobacterium OD₆₀₀ of infiltration was 2. Arrows indicate monomers (˜36 kDa, fragments 2 and 3) and dimers (66 kDa, fragment 1) of rH6 HA1 and a partially denatured product of ˜50 kDa (fragment 4).

FIG. 26: Protein sequence coverage for the plant produced recombinant H6 antigen (SEQ ID NO:7). Peptides with >95% confidence are in bold text, 50-95% in bold, italics and <50% confidence underlined. No peptides were identified for the regions of the sequences in italics only text.

FIG. 27: Mean values for replicates for indirect H7 ELISA with chicken sera. Coating antigen: rH7 HA1 at 1:150 dilution. Primary antibody: hyper-immune H7N7 chicken antiserum; SPF negative serum; in triplicate at 1:100 dilutions. Secondary antibody conjugate: commercial goat anti-chicken HRP, titrated from 1:1000 to 1:128000.

FIG. 28: Mean values for replicates for indirect H5 ELISA with ostrich sera. Coating antigen: rH5 HA1 at 1:50, 1:100, 1:150 and 1:200 dilutions, in duplicate. Primary antibody: Pooled ostrich H5 positive field antisera, medium titre (++), pooled ostrich H5 positive field antisera, low titre (+) and ostrich H5 negative antiserum. All at 1:100 dilution with 4 replicates. Secondary antibody conjugate: chicken anti-ostrich IgY conjugated to HRP at 1:50, 1:100, 1:1000 and 1:2000 dilutions, in duplicate.

FIG. 29: Mean values for replicates for double antigen sandwich (DAS) H5 ELISA. Coating antigen: rH5 HA1. Primary antibody: hyper-immune H5N2 chicken antiserum; Clade 2.3.4.4 field H5N8 antiserum; SPF negative antiserum, all at 1:100 dilutions (in duplicate). Conjugate: HRP-labelled rH5 HA1 (1:200 to 1:128000 titration).

FIG. 30: Recombinant genetic fusion rCHIR-AB1-HRP protein (SEQ ID NO:15).

FIG. 31: pEAQ-HT harbouring the rCHIR-AB1-HRP insert (SEQ ID NO:16) at the Age I/Xho I restriction sites.

FIG. 32: Immunoblot detection of HIS-tagged protein purification of rCHIR AB1 produced in N. benthamiana and probed with anti-HIS-HRP. Proteins were extracted five days post infiltration in a Bicine buffer. The Agrobacterium inoculum OD₆₀₀was 2. MW, Biorad precision plus protein WesternC standard; lane 1, flow through of Ni-TED column; lanes 2-3, Agrobacterium strain AGL-1 mediated CHIR AB1 production, 8 and 16 μl (103 and 206 ng protein, respectively); lanes 4-5, Agrobacterium strain GV3101::pM90 mediated production of CHIR AB1, 8 and 16 μl (267 and 535 ng protein, respectively) and lanes 6-7, Agrobacterium strain LBA4404 mediated production of CHIR AB1, 8 and 16 μl (113 and 227 ng protein, respectively). Arrows indicate monomers (˜57 kDa) of rCHIR-AB1.

FIG. 33: ELISA results evaluating the functionality of A) the commercially available anti-IgY HRP conjugate versus B) the plant produced crude extract conjugate (CHIR-AB1-HRP).

Sequence Listing

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:

SEQ ID NO:1—Amino acid sequence of the full-length H7-HA1 construct;

SEQ ID NO:2—Nucleotide sequence of the full-length H7-HA1 construct;

SEQ ID NO:3—Amino acid sequence of the truncated rH7-HA1 construct;

SEQ ID NO:4—Nucleotide sequence of the truncated rH7-HA1 construct;

SEQ ID NO:5—Amino acid sequence of the truncated rH5-HA1 construct;

SEQ ID NO:6—Nucleotide sequence of the truncated rH5-HA1 construct;

SEQ ID NO:7—Amino acid sequence of the truncated rH6-HA1 construct;

SEQ ID NO:8—Nucleotide sequence of the truncated rH6-HA1 construct;

SEQ ID NO:9—Amino acid sequence of the truncated rH9-HA1 construct;

SEQ ID NO:10—Nucleotide sequence of the truncated rH9-HA1 construct;

SEQ ID NO:11—Nucleotide sequence of pEAQ-HT forward primer;

SEQ ID NO:12—Nucleotide sequence of pEAQ-HT reverse primer;

SEQ ID NO:13—Nucleotide sequence of FSC5 forward primer;

SEQ ID NO:14—Nucleotide sequence of FSC5 reverse primer;

SEQ ID NO:15—Amino acid sequence of the rCHIR-AB1-HRP conjugate;

SEQ ID NO:16—Nucleotide sequence of the rCHIR-AB1-HRP conjugate.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

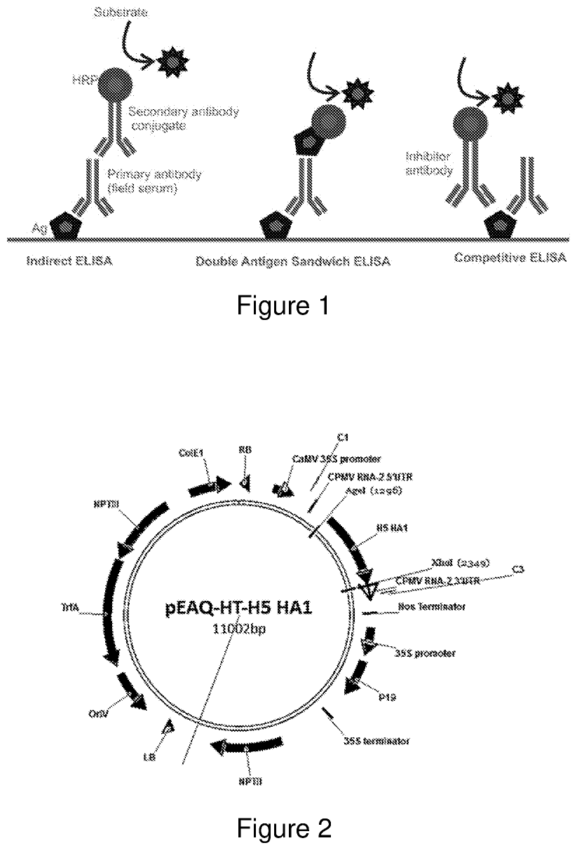

The present inventors have developed twelve different ELISA assays with plant produced antigens. This includes indirect ELISAs to detect anti-H5, -H6 -H7 and -H9 specific antibodies in chickens, a separate indirect ELISA to detect anti-H5, -H6, H7 and -H9-specific antibodies in ostriches, using a secondary chicken anti-ostrich IgY conjugate, and three additional ELISAs for H5, H6, H7 or H9 antibody detection in any species, by labelling the antigen itself with horseradish peroxidase, in a double antigen sandwich ELISA format (Table 1, FIG. 1).

TABLE 1
Applications of the technology (indirect and DAS ELISAs to detect
serotype-specific influenza A virus antibodies in poultry species) (nine products in total)

| | Recombinant coating antigen | | | |
|---|---|---|---|---|
| HRP conjugate | rH5-HA1 | rH6-HA1 | rH7-HA1 | rH9-HA1 |
| Commercial goat | Chicken-specific | Chicken-specific | Chicken-specific | Chicken-specific |
| α-chicken-HRP (e.g. | indirect ELISA to | indirect ELISA to | indirect ELISA to | indirect ELISA |
| Novex, Life | detect H5 | detect H6 | detect H7 | to detect H9 |
| Technologies) | antibodies | antibodies | antibodies | antibodies |
| OR rCHIR-AB1 HRP | | | | |
| Chicken α-ostrich-HRP | Ostrich-specific | Ostrich-specific | Ostrich-specific | Ostrich-specific |
| | indirect ELISA to | indirect ELISA to | indirect ELISA to | indirect ELISA |
| | detect H5 | detect H6 | detect H7 | to detect H9 |
| | antibodies | antibodies | antibodies | antibodies |
| rH5-HA1-HRP | Multi-species* | — | — | |
| | Double Antigen | | | |
| | Sandwich (DAS) | | | |
| | ELISA to detect | | | |
| | H5 antibodies | | | |
| rH6-HA1-HRP | — | Multi-species* | — | |
| | | DAS ELISA to | | |
| | | detect H6 | | |
| | | antibodies | | |
| rH7-HA1-HRP | — | — | Multi-species* | |
| | | | DAS ELISA to | |
| | | | detect H7 | |
| | | | antibodies | |
| rH9-HA1-HRP | | | | Multi-species* |
| | | | | DAS ELISA to |
| | | | | detect H9 |
| | | | | antibodies |

*Theoretically any species (human, horse, pig etc) for which validation data can be generated

The present invention relates to plant-produced avian influenza antigens (the “recombinant proteins”) or nucleic acids encoding the recombinant proteins and their uses.

A recombinant protein according to the invention includes, without limitation, a recombinant protein including the amino acid sequence of truncated rH5-HA1, rH6-HA1, rH7-HA1 or rH9-HA1, including an N-terminal barley amylase signal peptide or murine amylase signal peptide and a C terminal fusion to a thrombin cleavage site, a histidine tag and an endoplasmic reticulum targeting sequence.

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

The terms “nucleic acid” or “nucleic acid molecule” encompass both ribonucleic acids (RNA) and deoxyribonucleic acids (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

Accordingly, a “cDNA clone” refers to a duplex DNA sequence which is complementary to an RNA molecule of interest, and which is carried in a cloning vector. The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

In some embodiments, a recombinant protein of the invention may include, without limitation, a polypeptide including an amino acid sequence comprising a truncated rH5-HA1, rH6-HA1, rH7-HA1 or rH9-HA1 protein, including an N-terminal signal peptide selected from either barley amylase or murine amylase and a C-terminal thrombin cleavage site, histidine tag and an HDEL endoplasmic reticulum targeting sequence. Another embodiment of the invention includes, without limitation, nucleic acid molecules encoding the aforementioned recombinant proteins.

It will be appreciated by those of skill in the art that the Barley amylase signal peptide or the murine amylase signal peptide may be used interchangeably and direct the recombinant protein to the secretory pathway.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the antigenicity of the expressed recombinant protein or of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

In one embodiment of the invention, the recombinant proteins may be prepared by, for instance, inserting, deleting or replacing nucleic acids at any position of the nucleic acid molecule encoding the recombinant protein.

Those skilled in the art will appreciate that polypeptides, peptides or peptide analogues can be synthesised using standard chemical techniques, for instance, by automated synthesis using solution or solid phase synthesis methodology. Automated peptide synthesisers are commercially available and use techniques known in the art. Polypeptides, peptides and peptide analogues can also be prepared from their corresponding nucleic acid molecules using recombinant DNA technology.

In some embodiments, the nucleic acid molecules of the invention may be operably linked to other sequences. By “operably linked” is meant that the nucleic acid molecules encoding the recombinant proteins of the invention and regulatory sequences are connected in such a way as to permit expression of the recombinant proteins when the appropriate molecules are bound to the regulatory sequences. Such operably linked sequences may be contained in vectors or expression constructs which can be transformed or transfected into host cells for expression. It will be appreciated that any vector can be used for the purposes of expressing the recombinant proteins of the invention.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e. by human intervention. Recombinant nucleic acid constructs may be introduced into a host cell by transformation. Such recombinant nucleic acid constructs may include sequences derived from the same host cell species or from different host cell species.

The term “vector” refers to a means by which polynucleotides or nucleic acid sequences can be introduced into a cell. There are various types of vectors known in the art including plasmids, viruses, bacteriophages and cosmids. Generally polynucleotides or nucleic acid sequences are introduced into a vector by means of a cassette. The term “cassette” refers to a polynucleotide or nucleic acid sequence that is expressed from a vector, for example, the polynucleotide or nucleic acid sequences encoding the recombinant proteins of the invention. A cassette generally comprises a gene sequence inserted into a vector, which in some embodiments, provides regulatory sequences for expressing the polynucleotide or nucleic acid sequences. In other embodiments, the vector provides the regulatory sequences for the expression of the recombinant protein. In further embodiments, the vector provides some regulatory sequences and the nucleotide or nucleic acid sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication. For the purposes of the present invention an expression cassette is preferably used for the expression of the recombinant protein of the invention.

As mentioned the recombinant protein according to the invention includes, without limitation, a polypeptide including an amino acid sequence comprising a truncated rH5-HA1, rH6-HA1, rH7-HA1 or rH9-HA1 protein, including an N-terminal signal peptide selected from either barley amylase or murine amylase and a C-terminal thrombin cleavage site, histidine tag and an HDEL endoplasmic reticulum targeting sequence. Another embodiment of the invention includes, without limitation, nucleic acid molecules encoding the aforementioned recombinant proteins. It will be appreciated that an expression cassette encoding the recombinant protein also falls within the scope of the present invention.

The endoplasmic reticulum (ER) retention signal may include the amino acid sequence HDEL. Inclusion of an ER retention signal in the recombinant protein of the invention allows for endoplasmic reticulum retention of the expressed proteins. Other retention signals, for example KDEL, SEKDEL and the like, can also be used, which occur normally in animal and vegetable proteins localized in the ER, for the construction of the cassette.

The use of a plant expression system facilitates purification of the recombinant proteins via standard protein purification techniques. Typically, one sequence that can be added to a recombinant protein of the invention in order to assist in its purification is a histidine tag, or “His tag”. A histidine tag generally comprises a plurality of histidine residues. Passing the tagged protein over a column comprising a nickel N-(5-amino-1-carboxypentyl) iminodiacetic acid (Ni-NTA) agarose matrix can facilitate the isolation of the recombinant proteins comprising His tags.

In a preferred embodiment of the invention the recombinant proteins of the invention may be used for the detection of antibodies in a sample obtained for a subject in a diagnostic assay.

The following examples are offered by way of illustration and not by way of limitation.

### EXAMPLE 1

Experimental Design, Cloning, Transformation and Expression

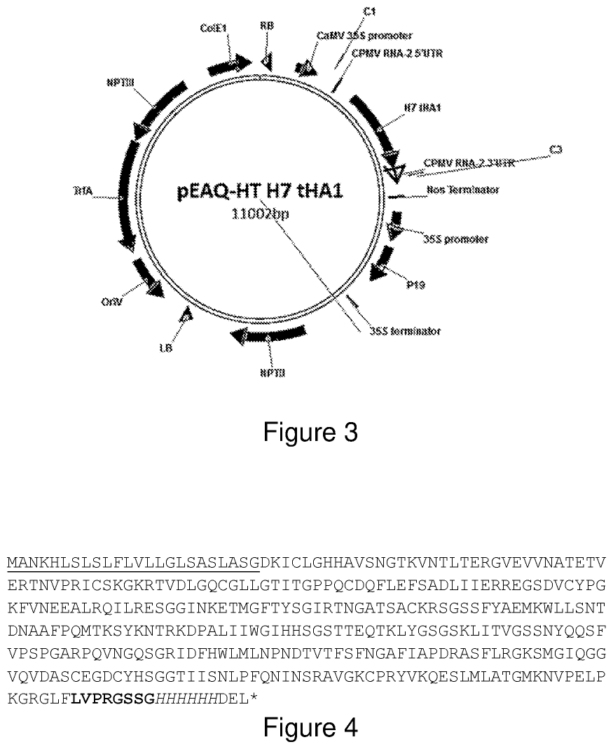

Plant codon-optimized avian influenza virus antigens of the H5 and H7 subtypes were designed and synthesized by Bio Basic Int. (Canada) with restriction sites for insertion into a cloning vector based on the cowpea Mosaic Virus (CPMV), pEAQ-HT (Sainsbury et al., 2009, 2010 and 2012). The nucleotide sequences was either Nicotiana benthamiana plant codon optimised or chicken (Gallus gallus) codon optimised by proprietary software of Bio Basic. The presence of the insert was confirmed with PCR amplification using generic molecular biology vector primers (two independent sets) (Table 2, FIGS. 2 and 3). Sequence validated constructs designated pEAQ-HT-H5-HA1 or pEAQ-HT-H7-HA1 were subsequently electroporated into a suitable Agrobacterium host strain, LBA4404, GV3101::pMK90 or AGL-1. Agrobacterium hosting the vector(s) of interest was syringe hand infiltrated into N. benthamiana and harvested 5-6 days after infiltration. pEAQ-HT empty vector and pEAQ-gfp without a His-tag served as negative controls.

TABLE 2
Two alternative sets of primer sequences
for vector insert validations (colony
PCR and sequencing.

| Target | | Primer | SEQ ID |
|---|---|---|---|
| Plasmid | Orientation | Sequence | NO |
| pEAQ-HT | Forward | 5′-ACTTGTTACGATTCT | SEQ ID |
| | primer | GCTGACTTTCGGCGG-3′ | NO: 11 |
| pEAQ-HT | Reverse | 5′-CGACCTGCTAAACAG | SEQ ID |
| | primer | GAGCTCACAAAGA-3′ | NO: 12 |
| FSC5 | Forward | 5′-GGTTTTCGAACTTGG | SEQ ID |
| | primer | AGAAA-3′ | NO: 13 |
| FSC5 | Reverse | 5′-AGAAAACCGCTCACC | SEQ ID |
| | primer | AAACATAGA-3′ | NO: 14 |

Protein design for the full-length recombinant hemagglutinin rH7-HA1 protein (SEQ ID NO:1, FIG. 4)) [Influenza A virus (A/ostrich/SouthAfrica/KVL/2012 (H7N1)), protein ID ALJ33344.1. It will be appreciated by those of skill in the art that either the Barley amylase signal peptide or the murine amylase signal peptide may be used interchangeably and will direct the recombinant protein to the secretory pathway. The theoretical cleavage of the signal peptide from full length or truncated HA1 with either the Barley amylase or murine signal peptide was verified using the SignalP 4.1 server. The plant Barley amylase signal peptide was selected and underlined in the sequence below, thrombin cleavage site typed in bold, C-terminal His-tag in italics and the C-terminal HDEL retention signal sequence as indicated:

(SEQ ID NO: 1)
MANKHLSLSLFLVLLGLSASLASGDKICLGHHAVSNGTKVN
TLTERGVEVVNATETVERTNVPRICSKGKRTVDLGQCGLLG
TITGPPQCDQFLEFSADLIIERREGSDVCYPGKFVNEEALR
QILRESGGINKETMGFTYSGIRTNGATSACKRSGSSFYAEM
KWLLSNTDNAAFPQMTKSYKNTRKDPALIIWGIHHSGSTTE
QTKLYGSGSKLITVGSSNYQQSFVPSPGARPQVNGQSGRID
FHWLMLNPNDTVTFSENGAFIAPDRASFLRGKSMGIQGGVQ
VDASCEGDCYHSGGTIISNLPFQNINSRAVGKCPRYVKQES
LMLATGMKNVPELPKGRGLELVPRGSSGHHHHHHDEL*.

Retaining of HA within the endoplasmic reticulum (ER) membrane of the host cell, signal peptide cleavage and protein glycosylation are co-translational events. It will be appreciated by those of skill in the art that the Barley amylase signal peptide or the murine amylase signal peptide may be used interchangeably and direct the recombinant protein to the secretory pathway].

The polynucleotide encoding the full-length recombinant rH7-HA1 protein, including the polynucleotides encoding the Barley amylase signal peptide, thrombin cleavage site, His-tag and ER retention signal was constructed with a sequence of (SEQ ID NO:2):

(SEQ ID NO: 2)
ATGGCAAACAAGCACCTAAGTCTGAGTCTGTTCCTGGTATT
GCTGGGACTTTCAGCTTCTTTGGCAAGTGGTGATAAGATCT
GTCTAGGTCATCATGCTGTAAGTAACGGAACAAAGGTAAAC
ACGCTAACGGAGAGAGGAGTTGAAGTTGTTAACGCAACAGA
GACGGTTGAAAGAACCAACGTTCCAAGAATCTGTAGTAAGG
GAAAGAGGACCGTTGATCTCGGACAATGTGGATTGTTGGGA
ACTATCACAGGACCACCACAATGTGATCAATTCCTCGAATT
CTCCGCTGATTTGATCATCGAACGACGAGAAGGATCAGATG
TTTGTTACCCTGGTAAGTTCGTTAACGAGGAAGCACTCAGA
CAAATTCTCAGAGAATCCGGTGGTATTAACAAGGAGACCAT
GGGTTTCACTTACTCCGGTATTAGAACCAATGGTGCAACCT
CAGCTTGCAAACGTAGTGGTTCATCTTTCTACGCAGAAATG
AAGTGGTTGCTTTCAAATACTGACAATGCCGCTTTTCCTCA
GATGACTAAGTCCTACAAGAATACACGGAAGGACCCTGCTT
TGATAATTTGGGGTATACACCACTCCGGATCAACAACAGAG
CAGACTAAACTTTATGGTAGCGGTAGCAAACTTATTACTGT
CGGTAGCAGCAATTATCAGCAGAGCTTTGTCCCTTCTCCGG
GAGCAAGGCCACAAGTTAATGGACAGTCTGGTCGTATTGAT
TTTCATTGGCTTATGCTTAATCCTAATGACACAGTCACTTT
TTCTTTTAATGGCGCCTTTATTGCCCCGGACAGGGCCTCTT
TTCTTAGGGGCAAATCTATGGGCATTCAGGGGGGGGTGCAA
GTGGATGCTTCTTGCGAGGGGGATTGCTATCATTCTGGCGG
CACTATAATTTCTAATTTACCATTTCAAAATATAAATTCAC
GGGCTGTGGGGAAATGCCCTAGGTATGTGAAACAGGAGTCA
CTTATGTTAGCTACAGGGATGAAAAATGTGCCCGAGTTACC
CAAAGGCCGTGGGTTATTTTTAGTTCCGCGGGGTTCATCTG
GGCATCATCACCATCACCACGATGAGTTATGA.

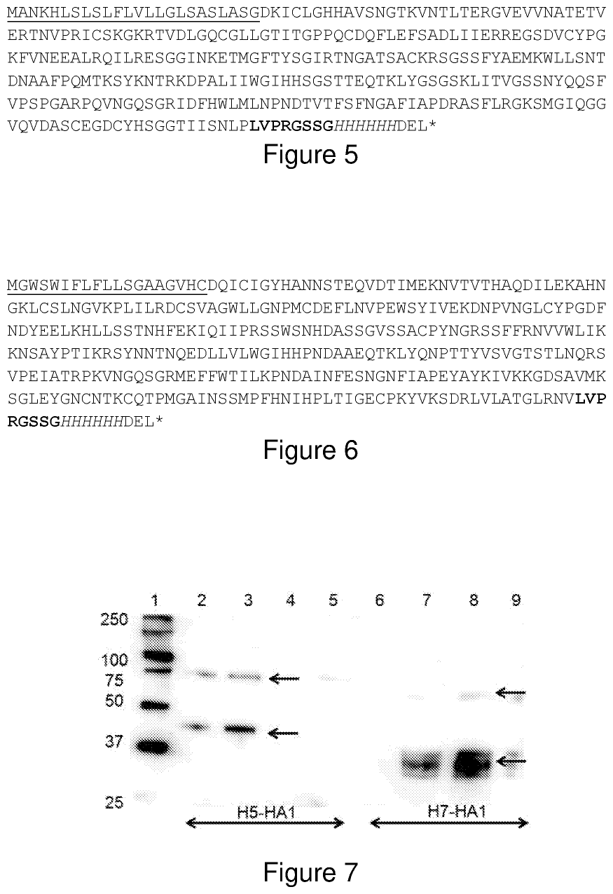

In a second experiment, the C-terminal 40 amino acids were removed which led to significant improvements in the expression of the recombinant protein, with no effect on antibody recognition (SEQ ID NO:3, FIG. 5). According to the literature, the important receptor binding and antigenic sites in HA1 are located between residues 157 and 226 (H3 numbering). The plant codon optimized nucleotide truncated rH7-HA1 sequence encoding the amino acid sequence below, cloned into pEAQ-HT, was used in all future experiments:

(SEQ ID NO: 3)
MANKHLSLSLFLVLLGLSASLASGDKICLGHHAVSNGTKVN
TLTERGVEVVNATETVERTNVPRICSKGKRTVDLGQCGLLG
TITGPPQCDQFLEFSADLIIERREGSDVCYPGKFVNEEALR
QILRESGGINKETMGFTYSGIRTNGATSACKRSGSSFYAEM
KWLLSNTDNAAFPQMTKSYKNTRKDPALIIWGIHHSGSTTE
QTKLYGSGSKLITVGSSNYQQSFVPSPGARPQVNGQSGRID
FHWLMLNPNDTVTFSENGAFIAPDRASFLRGKSMGIQGGVQ
VDASCEGDCYHSGGTIISNLPLVPRGSSGHHHHHHDEL*

The polynucleotide encoding the truncated recombinant rH7-HA1 construct, including the polynucleotides encoding the Barley amylase signal peptide, thrombin cleavage site, His-tag and ER retention signal was constructed with a sequence of (SEQ ID NO:4):

(SEQ ID NO: 4)
ATGGCAAACAAGCACCTAAGTCTGAGTCTGTTCCTGGTATT
GCTGGGACTTTCAGCTTCTTTGGCAAGTGGTGATAAGATCT
GTCTAGGTCATCATGCTGTAAGTAACGGAACAAAGGTAAAC
ACGCTAACGGAGAGAGGAGTTGAAGTTGTTAACGCAACAGA
GACGGTTGAAAGAACCAACGTTCCAAGAATCTGTAGTAAGG
GAAAGAGGACCGTTGATCTCGGACAATGTGGATTGTTGGGA
ACTATCACAGGACCACCACAATGTGATCAATTCCTCGAATT
CTCCGCTGATTTGATCATCGAACGACGAGAAGGATCAGATG
TTTGTTACCCTGGTAAGTTCGTTAACGAGGAAGCACTCAGA
CAAATTCTCAGAGAATCCGGTGGTATTAACAAGGAGACCAT
GGGTTTCACTTACTCCGGTATTAGAACCAATGGTGCAACCT
CAGCTTGCAAACGTAGTGGTTCATCTTTCTACGCAGAAATG
AAGTGGTTGCTTTCAAATACTGACAATGCCGCTTTTCCTCA
GATGACTAAGTCCTACAAGAATACACGGAAGGACCCTGCTT
TGATAATTTGGGGTATACACCACTCCGGATCAACAACAGAG
CAGACTAAACTTTATGGTAGCGGTAGCAAACTTATTACTGT
CGGTAGCAGCAATTATCAGCAGAGCTTTGTCCCTTCTCCGG
GAGCAAGGCCACAAGTTAATGGACAGTCTGGTCGTATTGAT
TTTCATTGGCTTATGCTTAATCCTAATGACACAGTCACTTT
TTCTTTTAATGGCGCCTTTATTGCCCCGGACAGGGCCTCTT
TTCTTAGGGGCAAATCTATGGGCATTCAGGGGGGGGTGCAA
GTGGATGCTTCTTGCGAGGGGGATTGCTATCATTCTGGCGG
CACTATAATTTCTAATTTACCATTAGTTCCGCGGGGTTCAT
CTGGGCATCATCACCATCACCACGATGAGTTATGA.

Protein design for a truncated version of the recombinant truncated hemagglutinin rH5-HA1 protein (SEQ ID NO:5, FIG. 6), [partial influenza A virus (A/ostrich/South Africa/C42 KF/2012(H5N2))] protein ID ALJ33273.1. The cleavage peptide PQRRKKRGLF of H5-HA1 was removed. The murine signal peptide is underlined, thrombin cleavage site in bold, C-terminal His-tag indicated in italics and C-terminal HDEL retention signal sequence are indicated:

(SEQ ID NO: 5)
MGWSWIFLELLSGAAGVHCDQICIGYHANNSTEQVDTIMEK
NVIVTHAQDILEKAHNGKLCSLNGVKPLILRDCSVAGWLLG
NPMCDEFLNVPEWSYIVEKDNPVNGLCYPGDFNDYEELKHL
LSSINHFEKIQIIPRSSWSNHDASSGVSSACPYNGRSSFFR
NVVWLIKKNSAYPTIKRSYNNINQEDLLVLWGIHHPNDAAE
QTKLYQNPITYVSVGISTLNQRSVPEIATRPKVNGQSGRME
FFWTILKPNDAINFESNGNFIAPEYAYKIVKKGDSAVMKSG
LEYGNCNTKCQTPMGAINSSMPFHNIHPLTIGECPKYVKSD
RLVLATGLRNVLVPRGSSGHHHHHHDEL*.

The polynucleotide encoding the truncated recombinant HA1 H5 construct, including the polynucleotides encoding the murine amylase signal peptide, thrombin cleavage site, His-tag and ER retention signal was constructed with a sequence of (SEQ ID NO:6):

(SEQ ID NO: 6)
ATGGGATGGAGTTGGATATTCCTGTTCCTACTATCCGGTGC
AGCTGGAGTACATTGTGATCAAATATGTATCGGATACCATG
CTAATAACTCCACTGAACAGGTAGATACGATCATGGAAAAG
AACGTAACGGTCACTCATGCACAAGATATCCTGGAAAAGGC
ACATAACGGTAAGCTATGTTCTCTGAACGGTGTTAAGCCAC
TTATCTTGAGAGATTGTTCCGTAGCAGGTTGGCTTCTTGGA
AATCCTATGTGTGATGAATTCTTAAACGTCCCTGAATGGTC
TTACATCGTTGAAAAGGATAACCCTGTTAACGGACTGTGTT
ACCCAGGTGATTTCAACGATTACGAAGAACTCAAGCATCTC
CTCTCTTCTACAAACCATTTCGAAAAGATACAGATTATTCC
CCGGAGTTCCTGGTCTAACCATGATGCAAGTAGCGGAGTTT
CAAGTGCATGCCCTTATAATGGAAGAAGTTCTTTCTTTCGA
AACGTGGTTTGGCTCATTAAGAAGAATAGCGCTTACCCAAC
AATTAAGCGTAGCTACAATAATACCAATCAGGAGGACTTGC
TTGTCTTGTGGGGAATTCACCACCCAAATGACGCTGCTGAA
CAAACTAAGTTGTATCAGAATCCGACCACTTATGTGAGCGT
TGGTACATCAACTTTGAATCAGAGAAGCGTTCCTGAGATTG
CTACACGACCAAAAGTTAATGGTCAATCAGGAAGAATGGAG
TTTTTTTGGACCATTCTTAAGCCCAATGACGCCATTAATTT
TGAGTCAAATGGGAATTTTATTGCCCCAGAGTATGCTTATA
AGATAGTGAAAAAAGGCGACAGTGCCGTGATGAAATCAGGC
TTGGAGTATGGTAATTGCAATACCAAATGCCAAACACCTAT
GGGCGCCATTAATTCTTCAATGCCGTTTCACAATATACACC
CTTTGACTATTGGGGAGTGCCCAAAATATGTCAAATCTGAC
CGTCTTGTGCTTGCTACAGGGCTTAGGAATGTGTTAGTTCC
GCGGGGTTCATCTGGGCATCATCACCATCACCACGATGAGT
TATG.

rH5-HA1 and rH7-HA1 (N. benthamiana codon-optimized) were independently cloned into pEAQ-HT (AgeI/XhoI cloning sites).

### EXAMPLE 2

Expression and Purification of Plant Produced Influenza Hemagglutinin

Genes synthesized by Bio Basic were cloned into pEAQ-HT by restriction digest (Age I/Xho I) and transformed into electro competent Escherichia coli DH1OB cells (1.8 kV, 200 Ω and 25 μF using a BIORAD Pulse Controller), resuspended in 500 μl SOC medium (0.5% w/v yeast extract, 2% w/v tryptone, 10 mM NaCl, 2.5 mM KCl, 10 mM MgCl₂, 10 mM MgSO₄, 20 mM glucose), and placed on a rotor shaker at 37° C. for an hour to recover before plated on Luria agar (10 g/L NaCl, 5 g/L yeast extract, 10 g/L Tryptone, 15 g/L agar (Oxoid)) plates, with the appropriate antibiotic (50 mg L-1 Kanamycin (Sigma)). Single colonies were grown overnight (37° C. at 175 rpm) in Luria broth (LB, 10 g/L NaCl, 5 g/L yeast extract, 10 g/L tryptone). DNA was isolated using the Zyppy™ plasmid miniprep kit (Epigenetics Company), colony PCR validated and DNA sequences were verified via dideoxy Sanger DNA sequencing (Inqaba Biotechnical Industries (Pty) Ltd).

Sequence validated gene inserts into the pEAQ-HT expression vector were subsequently electroporated into Agrobacterium tumefaciens LBA4404 (1.44 kV, 200 Ω and 25 μF). One nanogram of the recombinant pEAQ plasmid, purified from bacterial cells, was transformed into 60 μl electrocompetent LBA4404. Similarly pEAQ-HT void of an insert and pEAQ-HT-gfp were also electroporated into Agrobacterium and served as negative and positive controls, respectively. pEAQ-HT-gfp vector containing the green fluorescent protein gene (gfp) served as positive control. The product was resuspended in Luria broth medium, and placed on a rotor shaker at 30° C. for three hours to recover before plated on selective medium (20 mg/L streptomycin, 50 mg/L kanamycin, 25 mg/L rifamycin). Single colonies were grown overnight (28° C. at 175 rpm) and a minipreparation of DNA was once more electroporated into E. coli to validate sequence integrity. Agrobacterium containing pEAQ-HT with the sequence validated inserts were stored in glycerol stocks at −80° C. for tobacco plant infiltration. Constructs were sequenced (Inqaba Biotech) using the primer pairs designed for pEAQ-HT (Table 2).

Agrobacterium strain LBA4404 containing pEAQ-HT with inserts of choice were streaked on fresh LA plates containing rifampicin (25 mg/L), streptomycin (20 mg/L) and kanamycin (50 mg/L). The cultures were scraped from the plate and subsequently shake grown in liquid YMB media (0.1% yeast extract, 1% mannitol, 1.7 mM NaCl, 0.8 mM MgSO₄ and 2.2 mM K₂HPO₄) overnight at 28° C. at 175 rpm. Cultures were harvested at 8000 rpm for 7 minutes and resuspended in infiltration media (100-200 μM acetosyringone, 10 mM MES, 10 mM MgSO₄ at pH 5.6). The final OD₆₀₀ of the inoculum varied from 0.5 to 2 (Table 3). N. benthamiana plants (5-7 weeks old) were syringe hand infiltrated with individual Agrobacterium harbouring pEAQ-HT with the genes of choice.

Five to six days post infiltration (dpi) fresh leaf material was harvested and extracted in a Tris-HCl, pH 7.4 or bicine buffer (50 mM bicine, pH 8.4; 20 mM NaCl, 0.1% sodium laroylsarcosine, 1 mM dithiothreitol) in a ratio of 1:3 adding protease inhibitor cocktail (Sigma P2714). Fresh leaf material was harvested and blended in the described buffers independently. Crude extracts were filtered through two layers of Miracloth and the plant lysate centrifuged at 10 000 rpm for 15 minutes at 4° C. to remove cell debris. The supernatants were collected and equal volumes of the lysate subjected to Ni-TED His-tag purification (Macherey-Nagel, Protino 2000® packed columns) according to the manufacturer's recommendation. Eluted fractions were pooled (5 ml of each extract) and concentrated to 200 μl using Vivaspin® 6 (Polyethersulfonem PES membrane 3,000 MWCO; Sartorius Stedim Biotechnology, Catalogue number VS0691) according to the manufacturer's recommendations in a swing bucket (4 000 g×30 minutes).

SDS-PAGE and Immunoblot Detection

Proteins (15 μl per well) were subjected to TGX-Stain free chemiluminescent 10% acrylamide kit (Biorad Cat #161-0185) and using the precision plus protein WesternC standards (Biorad Cat #161-0183). The PVDF membrane was cut to the size of the unstained polyacrylamide gel and soaked firstly in methanol followed by brief washing in water, then immersion in transfer buffer (20% methanol, 1× Tris glycine SDS (Biorad, #1610732)). Extra thick blot filter papers (Bio-Rad) were also soaked in transfer buffer prior to the assembly of the semi-dry blotting system (Bio-Rad). Protein samples were transferred onto a PVDF membrane using a semi-dry blotting system (Biorad Trans-blot turbo) at 1 mA, 25 volts for 30 minutes. The membrane was blocked in 1× PBS buffer supplemented with 0.1% (v/v) Tween-20® and 3% (w/v) Bovine serum albumin (Fraction V) overnight at room temperature. The membranes were subsequently incubated with monoclonal anti-poly Histidine peroxidase conjugate (Sigma A7058 1VL at 1:1500) and Strep-Tactin HRP-conjugate (BIORAD 1610381 (1:5000) for 2 hours to detect the antigens and molecular marker, respectively. The membrane was washed three times in PBS Tween-20® buffer (PBS-T) for 10 minutes each. Finally, the membrane was subjected to detection with the Clarity™ Western ECL substrate (Biorad) and visualized with the ChemiDoc™ MP Imaging system (Biorad) according to the manufacturer's guidelines.

Mass Purification of H7 and H5

Six days post infiltration (dpi) fresh leaf material was harvested and blended using a commercial blender in a Bicine buffer as described above. A clear lysate was prepared by centrifuging at 12 000 rpm for 40 minutes in a JA14 rotor using a Beckman Coulter Avanti J-26 XPI centrifuge. The supernatants were collected in a Schott bottle and subjected to immobilized metal ion affinity chromatography, with nickel as the metal ion. The column used was a 5 ml bed volume Ni-TED resin packed in an XK16 column. Bound protein was eluted isocratically with 5 bed volumes of imidazole-containing buffer. All chromatographic operations for the purification of polyhistidine-tagged rH7-HA1 and rH5-HA1 proteins were conducted on an Akta Avant 150 instrument operated via Unicorn 6 software. Eluted fractions were pooled and concentrated using Vivaspin® 15 columns (Polyethersulfonem PES membrane; Sartorius Stedim Biotechnology, Catalogue number VS15T01) in a swing bucket (4 000 g×60 minutes). The concentrate was dialyzed against 2 L PBS buffer (140 mM NaCl, 1.5 mM KH₂PO₄, 10 mM Na₂HPO₄, 2.7 mM KCl, pH 7.4) at 4° C. and the buffer exchanged twice during 16 hours. Protein concentration was determined using the Micro BCA protein assay kit (Catalogue number 23235, Thermo Scientific).

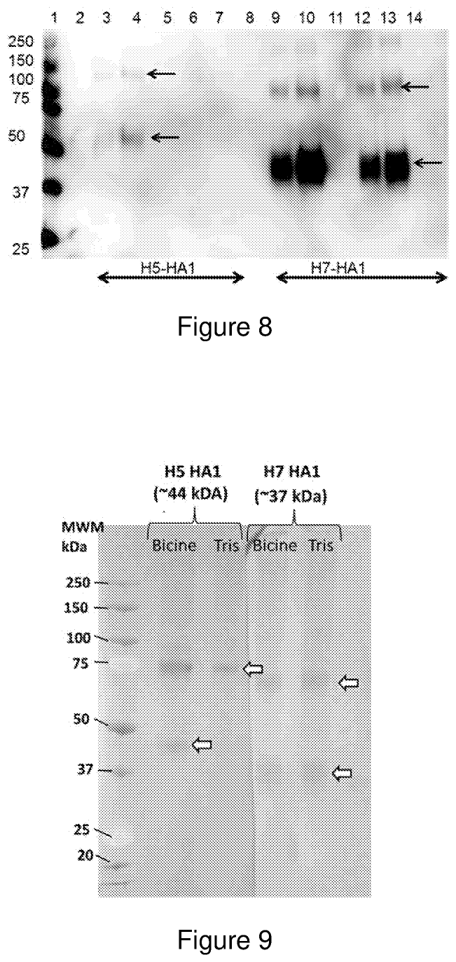

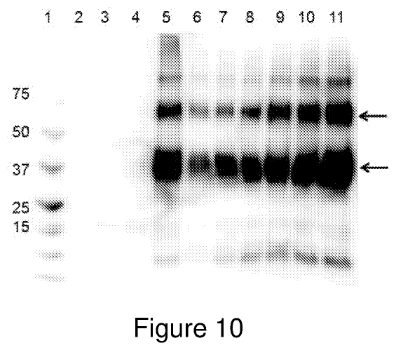

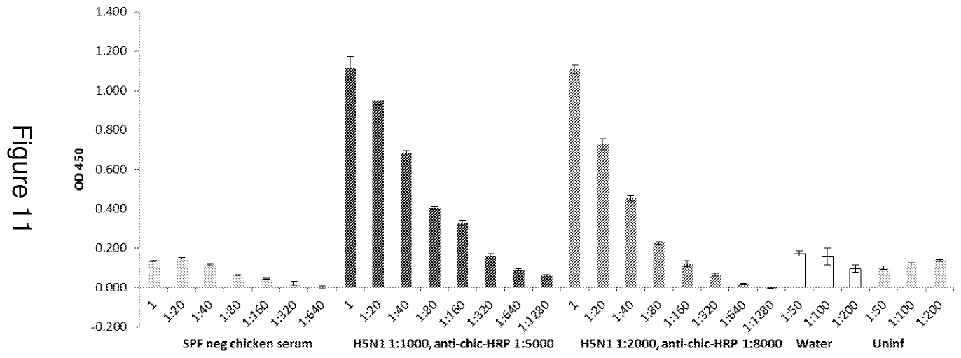

Target proteins were extracted as described and subjected to SDS-PAGE and Western blot analysis for detection (FIG. 7). Proteins purified were also subjected to Immunoblot detection using the anti-HIS-HRP antibody (FIG. 8). Subsequently, the protein integrity of rH5-HA1 and rH7-HA1 were validated using chicken anti-H5N2 and anti-H7N7 antibodies (FIG. 9). Preliminary results indicated that approximately 10 and 30 mg of rH5-HA1 and rH7-HA1 proteins, respectively, were produced in tobacco leaves. Mass purification resulted in 77 to 96 mg of rH7-HA1 protein from freshly harvested leaf material and directly purified using the AKTA Avant (Table 3) and once more validated by immunoblotting (FIG. 10). Frozen material resulted in much lower yields (Table 3).

Both rH7-HA1 and rH5-HA1 antigens were successfully expressed in N. benthamiana facilitating mammalian-like glycosylation (dXT/FT) (Strasser et al., 2008) plants and purified. Mass production and purification of both rH5-HA1 and rH7-HA1 resulted in >70 mg purified product per kilogram of fresh leaf tissue. Leaf tissue frozen before metal ion chromatography purification, resulted in approximately 50% and up to a 70% reduction in yield.

TABLE 3
Summary table of H5 and H7 antigens produced in N. benthamiana
facilitating mammalian-like glycosylation (dXT/FT). Antigens were
purified from fresh leaf material or * frozen leaf material.

| | | | | | | | mg HA |
|---|---|---|---|---|---|---|---|
| | | Avian | | | grams | Ni-TED elute | protein per |
| Date | Agrobacterial | influenza | | | leaf | (mg) | kg leaf |
| produced | strain | antigen | DPI | OD₆₀₀ | material | dialysed | tissue |
| Mar. 18, 2016 | LBA4404 | rH7-HA1 | 6 | 0.72 | 60 | 5.81 | 96.91 |
| Jun. 14, 2016 | LBA4404 | rH5-HA1 | 6 | 0.6 | 82.55 | 2.07 | 40* |
| Jun. 14, 2016 | LBA4404 | rH7-HA1 | 6 | 0.56 | 53 | 1.04 | 30* |
| Jul. 7, 2016 | LBA4404 | rH7-HA1 | 6 | 0.53 | 116 | 6.31 | 77.30 |
| Aug. 17, 2017 | LBA4404 | rH7-HA1 | 5 | 1.89 | 167 | 11.56 | 69.00 |
| Oct. 1, 2017 | AGL-1 | rH5-HA1 | 5 | 1.56 | 214.42 | 18.21 | 84.93 |

Vacuum Infiltration and Production of H5 Antigens

In short, vacuum infiltration (OD₆₀₀ of 0.4) at 30-50 mbar was conducted. Plant leaf material was harvested five days after infiltration and extracted in PBS (18 mM KH₂PO₄, 100 mM Na₂HPO₄, 27 mM KCl, 140 mM NaCl) buffer with the addition of 0.04% metabisulfite and protease inhibitor. Plant extract was clarified as described before. Chromatographic operations for the PBS purification of polyhistidine-tagged H5 HA1 proteins were conducted on an Akta Avant 150 instrument operated via Unicorn 6 software. Eluted fractions were pooled and concentrated using Vivaspin® 15 columns. The product was dialysed and quantified as described before. Vacuum infiltration and production of H5 antigen transiently expressed in N. benthamiana facilitating mammalian-like glycosylation (ΔXT/FT) plants resulted in 30 milligram of partially purified H5 antigen per kilogram of leaf tissue.

Enzyme-Linked Immunosorbent Assay (ELISA)

Fifty microliter (50 μl) volumes of H5 antigen, serially diluted in PBS were coated into the wells of 96-well Maxisorp® Nunc-Immunoplates, with overnight incubation at 4° C. Plates were washed thrice with 300 μl of PBS-T buffer. Two hundred microliters per well of casein based blocking buffer was incubated for two hours at room temperature before being washed as before. Chicken H5N2 primary polyclonal antiserum (1:1000 or 1:2000) was diluted in blocking buffer, was added as 50 μl volumes into wells and incubated for two hours. After a triplicate wash, anti-chicken-HRP (1:5000 or 1:8000) secondary antibody was added as 50 μl volumes into wells and incubated for two hours. After a final wash step, 50 μl of 3,3′,5,5′-Tetrramethylbenzidine (TMB) substrate (Sigma) was added to each well. Plates were incubated at room temperature for 10 minutes before the reaction was stopped with the addition of an equal volume of 2 M H₂SO₄. Absorbance was read at 450 nm on a HiDEX sense microplate reader.

According to the ELISA results (FIG. 11), 0.15 microgram of the H5 protein product is sufficient to coat a single well for efficient detection. If this amount is indeed sufficient, 30 mg of partially purified H5 antigen product will be enough for 100 000 ELISA duplicate test samples. The product will be subjected to specificity and sensitivity testing using infected and non-infected poultry serum.

### EXAMPLE 3

Selection of a Suitable Agrobacterium Strain

In order to elevate expression levels of target avian influenza antigens, alternative Agrobacterium strains were harnessed. LBA4404, AGL-1 and GV3101::pMP90 Agrobacterium strains were compared as a vehicle to deliver the expression vector pEAQ-HT, harbouring the selected gene constructs, to the plant cells.

Agrobacterium strains LBA4404, GV3101::pMP90 or AGL-1 containing pEAQ-HT with inserts of choice were streaked on fresh LA plates with appropriate antibiotics: 25 mg/L rifampicin and 50 mg/L kanamycin supplemented with 20 mg/L streptomycin (LBA4404), 10 mg/L gentamycin (GV3101::pMP90) or 50 mg/L carbenicillin (AGL-1). The cultures were scraped from the plate and subsequently shake grown in either LB (GV3101 and AGL-1) or YMB (LBA4404) liquid media overnight at 28° C. at 175 rpm. Cultures were harvested at 8000 rpm for 7 minutes and resuspended in infiltration media (100-200 μM acetosyringone, 10 mM MES, 10 mM MgSO₄ at pH 5.6). The final OD₆₀₀ of each inoculum was approximately 2. N. benthamiana plants (5-7 weeks old) were syringe hand infiltrated with individual pEAQ-HT harbouring the genes of choice.

Five days post infiltration (dpi) fresh leaf material was harvested and extracted in ice cold Bicine buffer (50 mM bicine, pH 8.4; 20 mM NaCl, 0.1% sodium laroylsarcosine, 1 mM dithiothreitol) in a ratio of 1:3 adding protease inhibitor cocktail (Sigma P2714). The leaf material was extracted using a Matstone juicer followed by 60 seconds blending with an IKA Ultra-Turrax. The extract was filtered through two layers of cheesecloth before centrifuging at 8 000 g for eight minutes. The clarified lysate (25 ml of each extract) was subjected to metal ion affinity chromatography (Protino Ni-TED® 1 ml pre-packed columns, Macherey-Nagel), eluted with 5 ml elution buffer before concentration to 200 μl using Vivaspin® 6 (Polyethersulfonem PES membrane 3,000 MWCO; Sartorius Stedim Biotechnology, Catalogue number VS0691) according to the manufacturer's recommendations. The concentrated protein samples were subjected to SDS PAGE followed by immunoblot detection as described in Example 2.

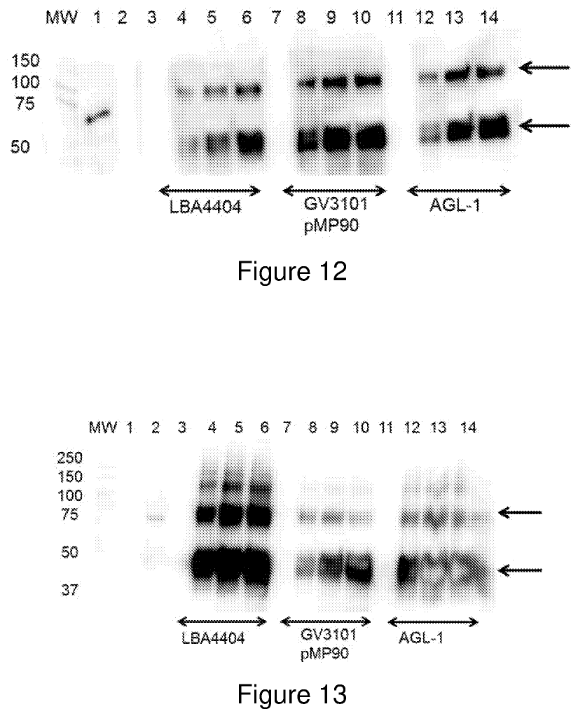

Expression of rH7-HA1 and rH5-HA1 antigens mediated by three independent Agrobacterium strains in N. benthamiana plants facilitating mammalian-like glycosylation (dXT/FT) were compared. Although relative similar expression levels (determined by immunoblot detection) were obtained harnessing the independent Agrobacterium strains, (FIGS. 12 and 13), AGL-1 and LBA4404 were chosen for future mass production of rH5-HA1 and rH7-HA1, respectively.

### EXAMPLE 4

Design and Cloning of Truncated rH9-HA 1

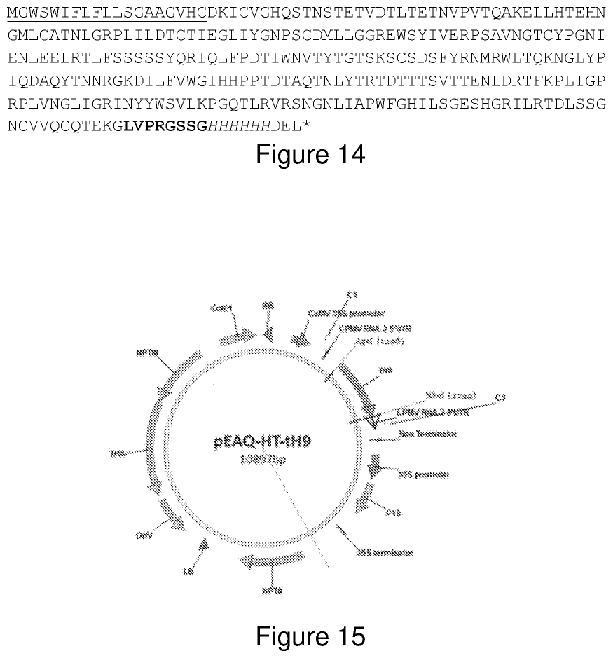

Gene design for a truncated version of the hemagglutinin HA1 H9 Influenza A virus protein (SEQ ID NO:9, FIG. 14) (A/chicken/Uganda/MUWRP-200192/2017(H9N2)) protein ID AVK87182.1 indicated below. The murine signal peptide is underlined, thrombin cleavage site in bold, C-terminal His-tag are in italics and C-terminal HDEL retention signal sequence are indicated. In order to investigate the impact of codon optimisation of nucleotide sequences encoding the target antigen production, the rH9-HA1 sequence was chicken (Gallus gallus) codon optimised and synthesized by BioBasic (Canada):

(SEQ ID NO: 9)
MGWSWIFLFLLSGAAGVHCDKICVGHQSTNSTETVDTLTET
NVPVTQAKELLHTEHNGMLCATNLGRPLILDTCTIEGLIYG
NPSCDMLLGGREWSYIVERPSAVNGTCYPGNIENLEELRTL
FSSSSSYQRIQLFPDTIWNVTYTGTSKSCSDSFYRNMRWLT
QKNGLYPIQDAQYTNNRGKDILFVWGIHHPPTDTAQTNLYT
RTDTTTSVTTENLDRTFKPLIGPRPLVNGLIGRINYYWSVL
KPGQTLRVRSNGNLIAPWFGHILSGESHGRILRTDLSSGNC
VVQCQTEKGLVPRGSSGHHHHHHDEL*.

The polynucleotide encoding the truncated recombinant rH9-HA1 protein, including the polynucleotides encoding the murine amylase signal peptide, thrombin cleavage site, His-tag and ER retention signal was constructed with a sequence of (SEQ ID NO:10) and cloned into pEAQ-HT (FIG. 15):

(SEQ ID NO: 10)
GACACCGGTATGGGCTGGAGCTGGATCTTTCTGTTCCTGCT
GAGCGGCGCCGCAGGAGTGCACTGCGATAAAATCTGCGTGG
GACACCAGAGCACAAACAGCACAGAGACAGTGGATACACTG
ACAGAGACTAACGTGCCTGTGACACAGGCCAAGGAACTGCT
GCACACAGAGCACAACGGCATGCTGTGCGCAACAAACCTGG
GCAGACCACTGATTCTTGATACATGCACAATTGAAGGTCTG
ATCTACGGAAACCCTAGCTGCGATATGCTGCTGGGAGGAAG
GGAGTGGTCATACATTGTGGAAAGACCTAGCGCCGTGAACG
GCACTTGCTACCCTGGAAACATTGAGAATCTGGAAGAACTG
AGAACCCTGTTCAGCAGCAGCTCTTCTTACCAGAGAATCCA
GCTGTTTCCTGATACAATTTGGAATGTGACATACACAGGCA
CATCTAAGAGCTGCAGCGATAGCTTTTATAGAAACATGAGG
TGGCTGACACAGAAAAACGGCTTATACCCCATCCAGGACGC
TCAGTATACAAATAATAGAGGAAAGGATATTCTGTTTGTGT
GGGGAATCCATCACCCCCCAACAGATACTGCACAGACAAAT
CTGTACACCAGAACTGATACTACAACATCTGTGACAACAGA
AAATCTGGATAGAACCTTTAAACCACTGATCGGACCAAGAC
CTCTGGTGAATGGACTGATTGGAAGAATTAATTACTACTGG
TCTGTGCTGAAACCTGGCCAGACACTGAGAGTGAGGAGCAA
CGGAAACCTGATCGCCCCCTGGTTTGGACACATTCTGTCTG
GAGAGAGCCACGGAAGAATCCTGAGAACAGATCTGAGCAGC
GGCAACTGCGTGGTGCAGTGTCAGACAGAGAAGGGACTGGT
GCCAAGAGGAAGCAGCGGCCACCACCACCACCATCACGATG
AGCTGTGACTCGAGGAC.

Expression and Small Scale Purification of Plant Produced Truncated H9 Influenza Hemagglutinin

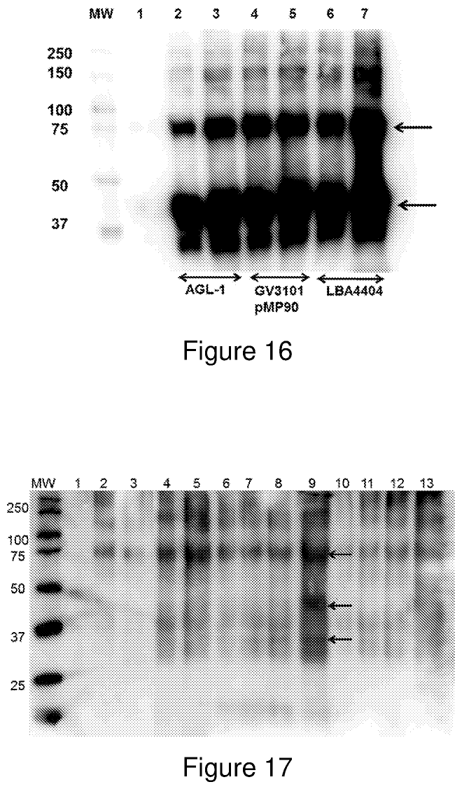

Small scale production, purification, SDS PAGE and His-tagged immunoblot detection of H9 protein is as described in Example 2. Identifying a suitable Agrobacterium strain is as described in Example 3. In short, proteins were extracted five days post infiltration using a Bicine buffer described before with the addition of 0.1% sodium laroylsarcosine and 1 mM dithiothreitol) in a ratio of 1:2 adding protease inhibitor cocktail (Sigma P2714). The truncated H9 protein was confirmed by His-tag immunoblotting. Subsequently, the membrane was subjected to detection with Clarity™ Western ECL substrate (Biorad) and visualized with the ChemiDoc™ MP Imaging system (Biorad) according to the manufacturer's guidelines (FIG. 16). An immunoblot using H9 anti-serum confirms the plant production of the H9 antigen using anti-H9N2 (1:1000) and anti-chicken-HRP (1:3000) (FIG. 17). Due to the antigen mismatch there is some background.

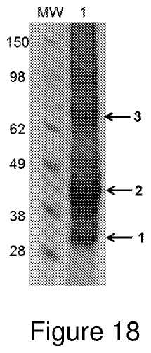

Preliminary results from small scale production of rH9-HA1 indicated that approximately 95, 34 and 47 mg of rH9-HA1 proteins were produced per kilogram tobacco leaves using AGL-1, GV3101 pM90 or LBA4404 as Agrobacterium strains, respectively. Partially purified H9 protein was subjected to sodium dodecyl sulfate polyacrylamide gel electrophoresis (SDS-PAGE) (FIG. 18) and the target protein fragments analyzed using liquid chromatography-mass spectrometry (LC MS/MS) based peptide sequencing.

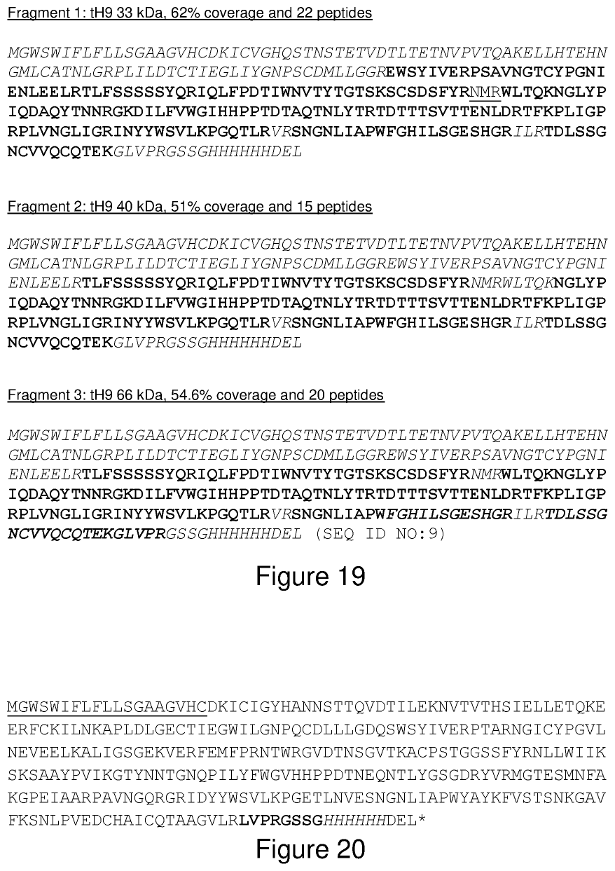

Both the immunoblots (anti-His and H9 anti-serum) and LC MS/MS based peptide sequencing confirms the plant based production of the recombinant H9 antigen (SEQ ID NO:9). LC-MS/MS based peptide sequence analysis for excised H9 antigen fragments from SDS PAGE gels were confirmed by 62%, 51% and 54.6% sequence coverage and 22, 15 and 20 peptides, respectively, identified with >95% confidence (FIG. 19). Peptides with >95% confidence are in black text and <50% confidence underlined. No peptides were identified for the regions of the sequence in grey text.

### EXAMPLE 5

Design and Cloning of Truncated rH6-HA 1

Gene design for a truncated version of the hemagglutinin rH6-HA1 protein (SEQ ID NO:7, FIG. 20), [H6 hemagglutinin [Influenza A virus (A/chicken/South Africa/BKR4/2012(H6N2))] protein ID ANZ03749.1 indicated below. The murine signal peptide is underlined, thrombin cleavage site in bold, C-terminal His-tag are in italics and C-terminal HDEL retention signal sequence are indicated. In order to investigate the impact of codon optimisation of nucleotide sequences encoding the target antigen production, the rH6-HA1 sequence was chicken (Gallus gallus) codon optimised and synthesized by BioBasic (Canada):

(SEQ ID NO: 7)
MGWSWIFLFLLSGAAGVHCDKICIGYHANNSTIQVDTILEK
NVIVIHSIELLETQKEERFCKILNKAPLDLGECTIEGWILG
NPQCDLLLGDQSWSYIVERPTARNGICYPGVLNEVEELKAL
IGSGEKVERFEMFPRNTWRGVDINSGVIKACPSIGGSSFYR
NLLWIIKSKSAAYPVIKGTYNNIGNQPILYFWGVHHPPDTN
EQNTLYGSGDRYVRMGTESMNFAKGPEIAARPAVNGQRGRI
DYYWSVLKPGETLNVESNGNLIAPWYAYKFVSTSNKGAVEK
SNLPVEDCHAICQTAAGVLRLVPRGSSGHHHHHHDEL*.

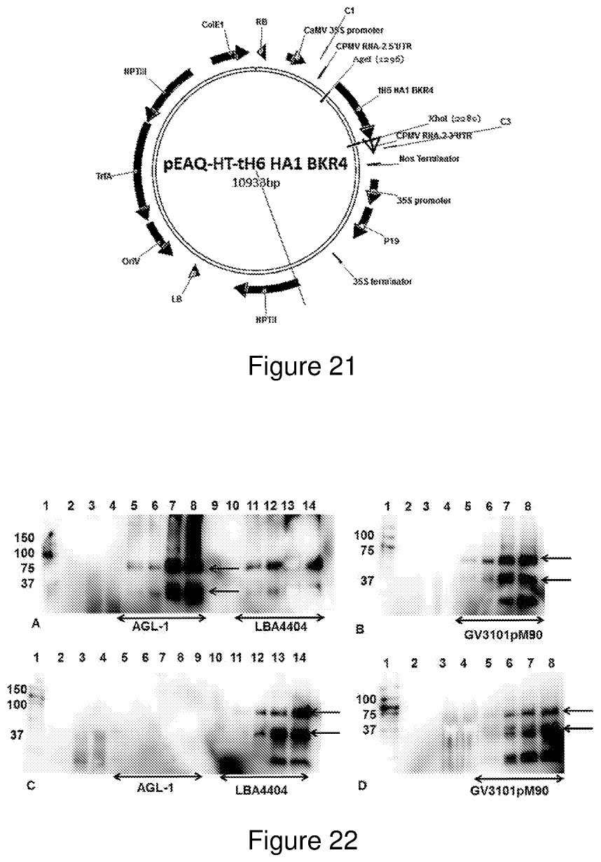

The polynucleotide encoding the truncated recombinant rH6-HA1 protein, including the polynucleotides encoding the murine amylase signal peptide, thrombin cleavage site, His-tag and ER retention signal (SEQ ID NO:8) was constructed with a sequence of (SEQ ID NO:8) and cloned into pEAQ-HT (FIG. 21):

(SEQ ID NO: 8)
ATGGGATGGAGCTGGATCTTCCTGTTCCTGCTGAGCGGCGC
TGCTGGAGTACACTGTGATAAAATCTGCATCGGATACCACG
CTAACAATAGCACAACCCAGGTGGATACAATCCTGGAGAAG
AACGTGACAGTGACTCACTCCATCGAGCTGCTGGAAACTCA
GAAAGAGGAAAGATTTTGCAAGATTCTGAACAAGGCTCCAC
TGGATCTGGGAGAGTGCACAATCGAAGGATGGATTCTGGGA
AATCCTCAGTGTGATCTGCTGCTGGGAGATCAGAGCTGGAG
CTACATCGTGGAGAGACCCACCGCCAGAAACGGCATCTGCT
ACCCCGGCGTGCTGAACGAGGTGGAGGAGCTGAAGGCCCTG
ATCGGCAGCGGCGAGAAGGTGGAGAGATTCGAGATGTTCCC
TAGGAACACATGGAGGGGCGTGGACACAAACAGCGGCGTGA
CAAAGGCCTGCCCCAGCAGCACCGGCGGCAGCAGCTTCTAC
AGAAACCTGCTGTGGATCATAAAGAGCAAGAGCGCCGCCTA
CCCCGTGATCAAGGGCACCTACAACAACACCGGCAACCAGC
CCATCCTGTACTTCTGGGGCGTGCACCACCCCCCCGACACC
AACGAGCAGAACACACTGTACGGAAGCGGCGATAGGTACGT
GAGAATGGGCACTGAATCCATGAATTTTGCTAAAGGACCTG
AGATAGCTGCTAGACCCGCCGTTAACGGCCAGAGAGGCAGA
ATCGATTACTACTGGAGCGTGCTGAAGCCCGGCGAGACACT
GAACGTGGAGTCTAACGGCAACCTGATCGCCCCTTGGTACG
CCTACAAGTTCGTGAGCACCAGCAACAAGGGAGCCGTGTTC
AAGAGCAACCTGCCCGTGGAGGATTGCCACGCCATCTGCCA
GACCGCCGCCGGCGTGCTGAGACTGGTGCCCAGGGGCAGCA
GCGGCCACCACCACCACCATCATGATGAACTGTGA.

Expression and Small Scale Purification of Plant Produced Truncated H6 Influenza Hemagglutinin

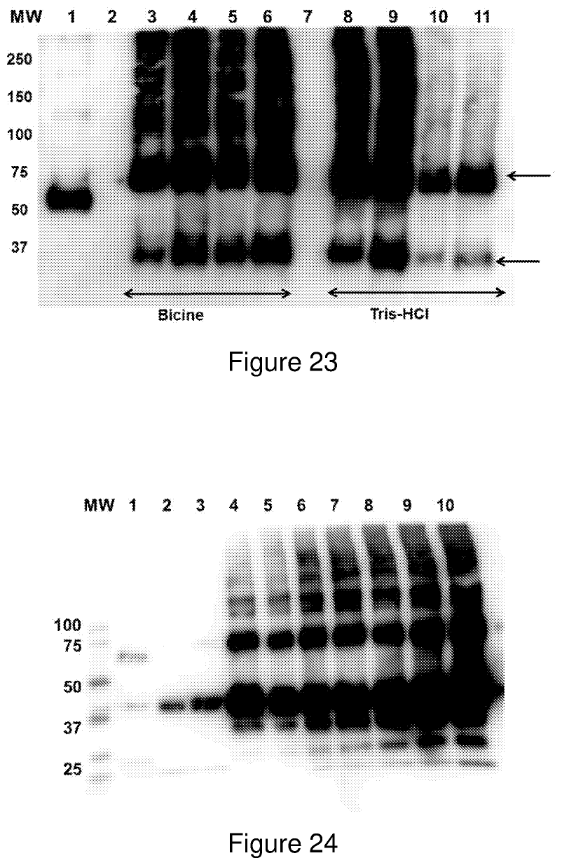

Small scale production, purification, SDS PAGE and His-tagged immunoblot detection of H6 protein is as described in Example 2. Identifying a suitable Agrobacterium strain is as described in Example 3. In short, proteins were extracted five days post infiltration using either a Bicine buffer described before or Tris-HCl buffer (50 mM Tris, 150 mM NaCl, 0.04% Na₂S₂O₅ sodium metabisulphite, pH 8.0) both with the addition of 0.1% sodium laroylsarcosine and 1 mM dithiothreitol) in a ratio of 1:2 adding protease inhibitor cocktail (Sigma P2714). In addition, to His-tag immunoblot confirmation of H6 production, detection of the actual intact rH6-HA1 protein was conducted using H6-specific antisera raised in chickens (1:650 dilution, Deltamune) followed by a goat anti-chicken-IgY horseradish peroxidase (HRP) conjugated secondary antibody (1:1,500 dilution; Novex Life Technologies). Subsequently, the membrane was subjected to detection with Clarity™ Western ECL substrate (Biorad) and visualized with the ChemiDoc™ MP Imaging system (Biorad) according to the manufacturer's guidelines (FIGS. 22 and 23).

Mass production and purification of H6 was as described in Example 2 with the following adjustments. Six days post infiltration (dpi) fresh leaf material was harvested and the clear lysate prepared by centrifuging at 9 500 rpm for 90 minutes in a JA10 rotor using a Beckman Coulter Avanti J-26 XPI centrifuge. The supernatants were collected in a 1 L Schott bottle and subjected to immobilized metal ion affinity chromatography (IMAC), with nickel as the metal ion. The column used was a 7 ml bed volume Ni-TED resin packed in a 010/10 column (GE Health). Bound protein was eluted isocratically with 5 bed volumes of imidazole-containing buffer. Eluted fractions were pooled and concentrated using Vivaspin® 15 columns (Polyethersulfonem PES membrane; Sartorius Stedim Biotechnology, Catalogue number VS15T01) in a swing bucket (4 000 g×60 minutes). The concentrate was dialyzed against 2 L PBS buffer (140 mM NaCl, 1.5 mM KH₂PO₄, 10 mM Na₂HPO₄, 2.7 mM KCl, pH 7.4) at 19° C. during 16 hours. Protein concentration was determined using the Micro BCA protein assay kit as before. Immunoblot detection was as described before and visualised in FIG. 24.

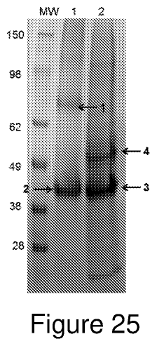

Preliminary results indicated that approximately 26 mg/kg of rH6-HA1 proteins was produced in tobacco leaves. Partially purified H6 protein was subjected to sodium dodecyl sulfate polyacrylamide gel electrophoresis (SDS-PAGE) (FIG. 25) and the target protein fragments analyzed using liquid chromatography-mass spectrometry (LC MS/MS) based peptide sequencing.

Liquid Chromatography-Mass Spectrometry LC MS/MS Based Peptide Sequencing Validation of H6

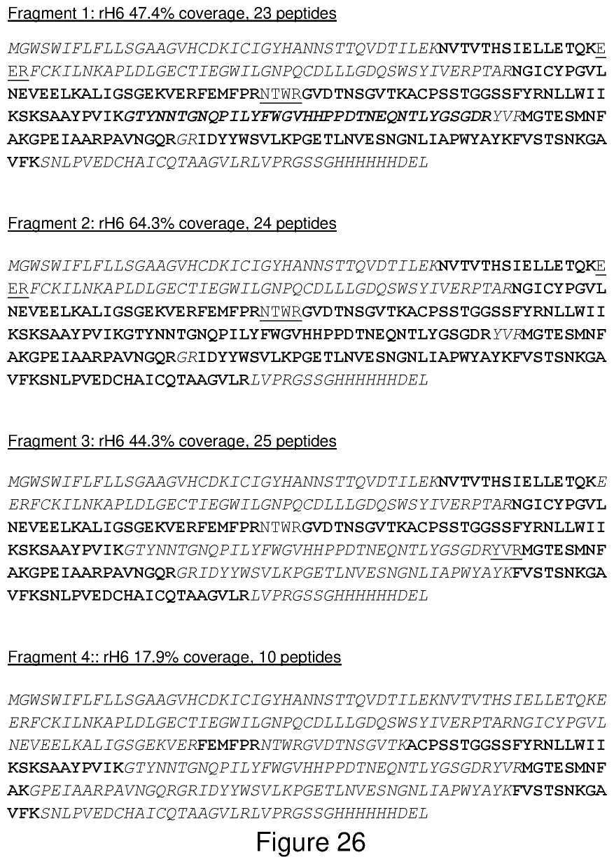

Protein bands of interest were in-gel trypsin digested as per the protocol described in (Shevchenko et al., 2007). In short, gel bands were destained using 50 mM NH₄HCO₃/50% MeOH followed by in-gel protein reduction (50 mM DTT in 25 mM NH₄HCO₃) and alkylation (55 mM iodoacetamide in 25 mM NH₄HCO₃). Proteins were digested over night at 37C.° using 5-50 μl, 10 ng/μl tryspin depending on the gel piece size. Digests were resuspended in 20 μl, 2% acetonitrile/0.2% formic acid and analysed using a Dionex Ultimate 3000 RSLC system coupled to an AB Sciex 6600 TipleTOF mass spectrometer. Peptides were first de-salted on an Acclaim PepMap C18 trap column (100 μm×2 cm) for 2 min at 15 μl/min using 2% acetonitrile/0.2% formic acid, than separated on Acclain PepMap C18 RSLC column (300 μm×15 cm, 2 μm particle size). Peptide elution was achieved using a flow-rate of 8 μl min⁻¹ with a gradient: 4-60% B in 15 min (A: 0.1% formic acid; B: 80% acetonitrile per 0.1% formic acid). An electrospray voltage of 5.5 kV was applied to the emitter. The 6600 TipleTOF mass spectrometer was operated in Data Dependent Acquisition mode. Precursor scans were acquired from m/z 400-1500 using an accumulation time of 250 ms followed by 30 product scans, acquired from m/z 100-1800 at 100 ms each, for a total scan time of 3.3 sec. Multiply charge ions (2+-5+, 400 -1500 m/z) were automatically fragmented in Q2 collision cells using nitrogen as the collision gas. Collision energies were chosen automatically as function of m/z and charge. Protein pilot v5 using Paragon search engine (AB Sciex) was used for comparison of the obtained MS/MS spectra with Uniprot Swissprot protein database. Proteins with threshold above ≥99.9% confidence were reported (FIG. 26).

Both the immunoblots and LC MS/MS based peptide sequencing confirms the plant produced production of the recombinant H6 antigen (SEQ ID NO:7). Extraction in the Bicine buffer resulted in a more pure rH6 HA1 product and protein sizes as anticipated (monomers at ˜36 kDa and dimers at ˜66 kDa) than obtained when extracting in a Tris-based buffer.

### EXAMPLE 6

Horseradish Peroxidase (HRP) Conjugate Production

Chicken α Ostrich HRP (Secondary Antibody Conjugate)

A fresh ostrich egg was obtained from Klein Karoo International Laboratory (Pty) Ltd, Oudtshoorn. The ostrich egg yolk was carefully removed without rupturing the yolk sac, and rinsed with distilled pure water to remove residual albumin. The yolk volume was measured and diluted tenfold with distilled water. After filtering through gauze to remove the yolk sac membrane, HCl was added to a final concentration of 3 mM, and the pH was adjusted to 5.0. Lipids were precipitated by overnight incubation at 4° C. The egg yolk was centrifuged at 8000 ×g for 10 mins in an Eppendorf 5804R centrifuge, and the clear supernatant was collected. One ml of the IgY was further purified with a HiTrap™ IgY HP column (GE Healthcare, Uppsala, Sweden), according to the recommended procedure. IgY was concentrated with a Vivaspin® 20 5,000 MWCO column (Sartorius), and the protein concentration of 2.9 mg/ml was determined using a Pierce BCA Protein Assay kit (Thermo Scientific).

Four point-of-lay Specific Pathogen Free (SPF) White Leghorn breed hens were obtained from AviFarms (Pty) Ltd, Pretoria. The hens were housed at the Poultry Research Unit at the Faculty of Veterinary Science, in floor pens with ad libitum access to food and water. Each hen was immunized with 3 μg of purified ostrich IgY, mixed in a 1:1 ratio with Montanide® ISA70 (Seppic, France). Each hen received three intra-muscular immunizations at day 0, 21 and 35. Eggs laid from a week after the last immunization onwards were collected. IgY was precipitated and quantified as described above. All animal procedures were conducted with approval of the University of Pretoria's Animal Ethics Committee, under project number v045-17.

Protein Coupling to HRP (Secondary Antibody Conjugate and DAS Labelled Antigen)

Chicken α-ostrich IgY, rH5-HA1 and rH7-HA1 (H6 pending) were coupled to HRP using a Lightning-Link® HRP Conjugation kit (Innova Biosciences, Cambridge, UK) according to the recommended procedure, in the 1:4 Ab:HRP ratio, overnight. After the incubation step with LL-quencher, and equal volume of BioStab Antibody Stabilizer (Sigma Life Science, Germany) was added, and the conjugates were stored at 4° C.

### EXAMPLE 7

Enzyme-Linked Immunosorbent Assay (ELISA)

One hundred microliter (100 μl) volumes of recombinant unlabelled antigen dilutions (rH5-HA1, rH6-HA1 or rH7-HA1) in carbonate-bicarbonate buffer (Sigma-Aldrich) were coated into the wells of 96-well Maxisorp Nunc-Immunoplates, with overnight incubation at 4° C. Plates were washed 3× with 300 μl of PBS-T buffer in a Bio-Rad ImmunoWash™ 1575 Microplate Washer.

One hundred microliters per well of blocking buffer (10% fat-free milk power in TST buffer) was incubated with shaking for one hour at 37° C. before being washed as before. Primary antibody was diluted 1:100 in blocking buffer, and added in 100 μl volumes into wells.

Primary antibodies consisted of known positive and negative antisera from chickens and ostriches. Primary chicken antibodies were obtained from standardized influenza A H5N1, H5N2, H6N2, H6N8, H7N1 and H7N7 chicken antisera which were purchased from Deltamune (Pty) Ltd, Pretoria. Additionally, primary ostrich antibodies were obtained from panels of H5, H6 and H7-specific and negative field ostrich sera provided by Deltamune (Pty) Ltd, Oudtshoorn. Strongly positive H5 and H7-specific ostrich sera were selected from the panels after screening with ID Screen® Influenza H5 Antibody Competition (FLUACH5-2P) and ID Screen® Influenza H7 Antibody Competition (FLUACH7-2P) kits, supplied by IDVet, France.

After incubation with shaking for one hour at 37° C., the plates were washed as before, and serial dilutions of the relevant recombinant antigen-HRP conjugate was added (refer to Table 1). After a final one hour incubation and wash step, 100 μl of 1-Step™Ultra TMB-ELISA substrate (Thermo Scientific, Rockford, Ill., USA) was added to each well. Plates were incubated at room temperature for 15 minutes before the reaction was stopped with the addition of an equal volume of 2 M H₂SO₄. Absorbance was read at 450 nm on a Bio-Rad iMark™ Microplate Reader.

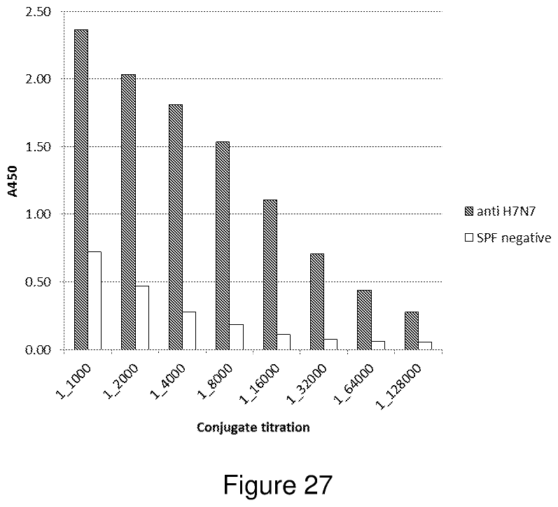

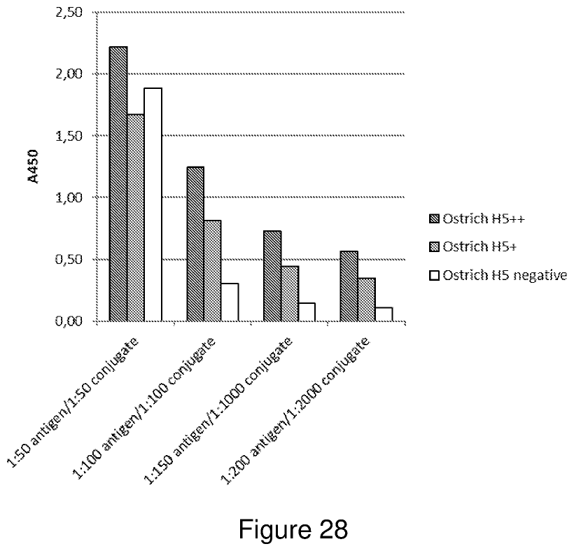

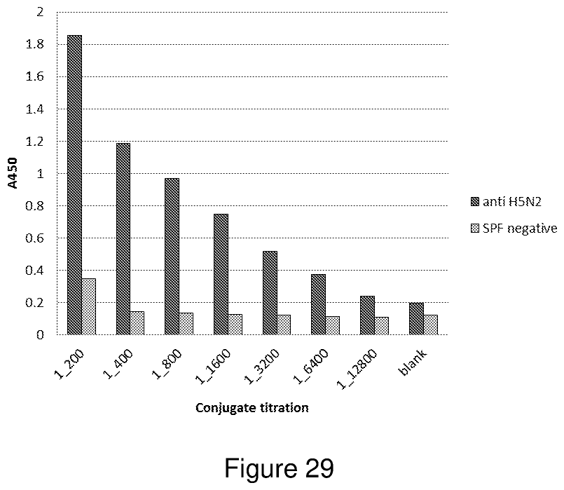

Optimal coating antigen, primary antibody and conjugate concentrations were determined by checkerboard titrations. Indirect H7 ELISA was performed with chicken sera (Table 4). Indirect H5 ELISA was performed with ostrich sera (Table 5) and double antigen sandwich (DAS) H5 ELISA was performed (Table 6). The mean values for replicates are presented in FIGS. 27-29.

TABLE 4
Summary table of indirect H7 ELISA with chicken sera.
Coating antigen: rH7 HA1 at 1:150 dilution. Primary
antibody: hyper-immune H7N7 chicken antiserum; SPF
negative serum; in triplicate at 1:100 dilutions.
Secondary antibody conjugate: commercial
goat anti-chicken HRP, titrated from 1:1000 to 1:128000.

| | chicken anti H7N7 | | | SPF negative | | |
|---|---|---|---|---|---|---|
| 1_1000 | 2.361 | 2.338 | 2.389 | 0.728 | 0.723 | 0.719 |
| 1_2000 | 2.06 | 2.01 | 2.022 | 0.449 | 0.441 | 0.515 |
| 1_4000 | 1.818 | 1.704 | 1.906 | 0.291 | 0.273 | 0.274 |
| 1_8000 | 1.545 | 1.514 | 1.545 | 0.157 | 0.193 | 0.203 |
| 1_16000 | 1.097 | 1.114 | 1.108 | 0.098 | 0.111 | 0.126 |
| 1_32000 | 0.689 | 0.722 | 0.708 | 0.074 | 0.079 | 0.077 |
| 1_64000 | 0.425 | 0.442 | 0.446 | 0.061 | 0.062 | 0.063 |
| 1_128000 | 0.271 | 0.281 | 0.283 | 0.059 | 0.057 | 0.057 |

TABLE 5
Summary table of indirect H5 ELISA with ostrich sera. Coating antigen: rH5
HA1 at 1:50, 1:100, 1:150 and 1:200 dilutions, in duplicate. Primary antibody: Pooled ostrich
H5 positive field antisera, medium titre (++), pooled ostrich H5 positive field antisera, low titre
(+) and ostrich H5 negative antiserum. All at 1:100 dilution with 4 replicates. Secondary
antibody conjugate: chicken anti-ostrich IgY conjugated to HRP at 1:50, 1:100, 1:1000 and
1:2000 dilutions, in duplicate.

| | Pooled H5 ++ ostrich field | | | | Pooled H5 + ostrich field | | | | H5 negative ostrich field | | | |
|---|---|---|---|---|---|---|---|---|---|---|---|---|
| | antisera | | | | antisera | | | | serum | | | |
| 1:50 antigen | 2.403 | 2.209 | 2.211 | 2.15 | 1.644 | 1.607 | 1.698 | 1.67 | 0.745 | 0.876 | 0.939 | 2.302 |
| 1:50 conjugate | 2.413 | 2.09 | 2.114 | 2.186 | 1.701 | 1.692 | 1.661 | 1.687 | 0.759 | 0.812 | 0.734 | 1.473 |
| 1:100 antigen | 1.33 | 1.179 | 1.215 | 1.226 | 0.832 | 0.782 | 0.788 | 0.845 | 0.251 | 0.263 | 0.247 | 0.291 |
| 1:100 conjugate | 1.367 | 1.184 | 1.22 | 1.228 | 0.829 | 0.814 | 0.822 | 0.815 | 0.274 | 0.267 | 0.257 | 0.31 |
| 1:150 antigen | 0.779 | 0.685 | 0.68 | 0.721 | 0.421 | 0.424 | 0.426 | 0.451 | 0.14 | 0.139 | 0.129 | 0.144 |
| 1:1000 conjugate | 0.838 | 0.707 | 0.711 | 0.728 | 0.47 | 0.454 | 0.442 | 0.479 | 0.147 | 0.138 | 0.135 | 0.148 |
| 1:200 antigen | 0.649 | 0.504 | 0.495 | 0.498 | 0.267 | 0.289 | 0.297 | 0.332 | 0.082 | 0.087 | 0.074 | 0.093 |
| 1:2000 conjugate | 0.671 | 0.583 | 0.586 | 0.516 | 0.327 | 0.397 | 0.421 | 0.418 | 0.118 | 0.116 | 0.114 | 0.126 |

TABLE 6
Summary table of double antigen sandwich (DAS) H5 ELISA.
Coating antigen: rH5 HA1. Primary antibody: hyper-immune
H5N2 chicken antiserum; Clade 2.3.4.4 field H5N8 antiserum;
SPF negative antiserum, all at 1:100 dilutions (in duplicate).
Conjugate: HRP-labelled rH5 HA1 (1:200 to 1:128000 titration).

| | H5N2 antiserum | | H5N8 antiserum | | SPF negative | |
|---|---|---|---|---|---|---|
| 1:200 | 2.019 | 1.691 | 0.477 | 0.416 | 0.357 | 0.345 |
| 1:400 | 1.276 | 1.094 | 0.213 | 0.215 | 0.129 | 0.165 |
| 1:800 | 1.03 | 0.91 | 0.188 | 0.196 | 0.132 | 0.14 |
| 1:1600 | 0.793 | 0.705 | 0.166 | 0.159 | 0.124 | 0.13 |
| 1:3200 | 0.569 | 0.465 | 0.127 | 0.132 | 0.118 | 0.126 |
| 1:6400 | 0.413 | 0.334 | 0.125 | 0.12 | 0.109 | 0.124 |
| 1:12800 | 0.262 | 0.219 | 0.118 | 0.112 | 0.114 | 0.108 |
| blank | 0.22 | 0.178 | 0.118 | 0.121 | 0.119 | 0.127 |

### EXAMPLE 8

Recombinant Production of a Peroxidase-Antigen Genetic Recombinant Protein in Plants

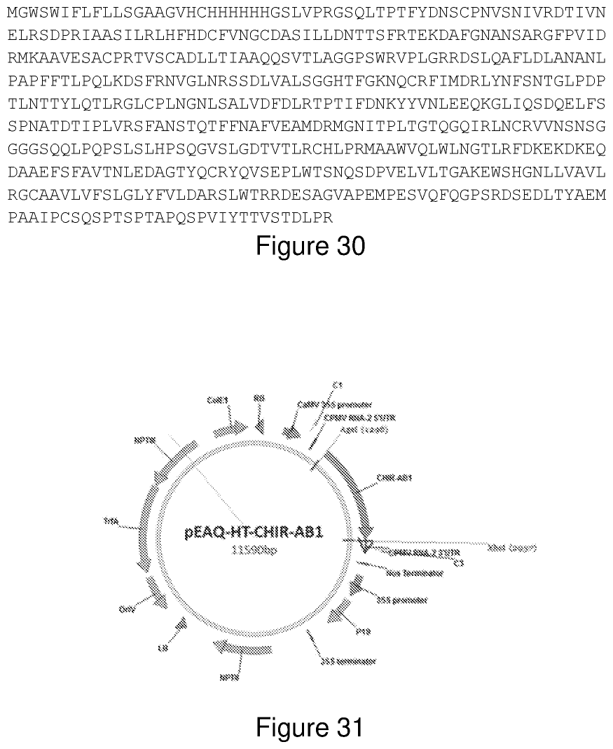

A reporter horseradish peroxidase (HRP) enzyme is required for the multispecies sandwich ELISA. Chemical conjugation with antibodies and antigens often results in low reproducibility and undefined stoichiometry. We designed a recombinant genetic fusion antigen-HRP and rCHIR-AB1-HRP (FIG. 30, SEQ ID NO:15) (Viertlboeck et al., 2007) which were produced in plants. The advantage of this approach is that it avoids the need for laborious and costly chemical conjugation. It is anticipated that the genetic fusion approach will result in conjugates with 1:1 stoichiometry, homogenous and reproducible molecules exhibiting functional activity of both the marker protein and the antigen/surface protein for cost-effective H5, H7, H6 and H9 diagnostic kits.

Design and Cloning of Plant Produced rCHIR-AB1-HRP as a Conjugate

An HRP conjugates was designed for immunoglobulin-like receptor CHIR-AB1 precursor [Gallus gallus] CHIR-AB1 AJ745094, Protein ID CAG33732.1 (Viertlboekck et al., 2007). The conjugate was chicken (Gallus gallus) codon optimised and synthesized by BioBasic (Canada).

The polynucleotide sequence encoding the rCHIR-AB1-HRP was cloned into a pEAQ-HT vector (FIG. 31).

Expression and Small Scale Purification of Plant Produced Conjugate

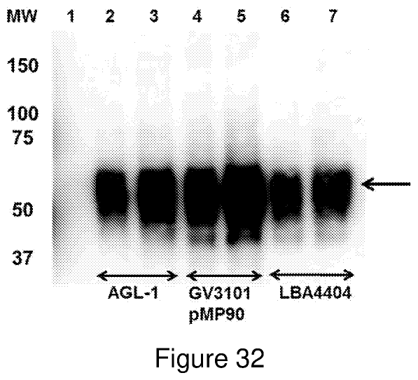

Small scale production, purification, SDS PAGE and His-tagged immunoblot detection of conjugate proteins was conducted as described in Example 2. Identifying a suitable Agrobacterium strain is as described in Example 3. In short, proteins were extracted 4-5 days post infiltration using a Bicine buffer described with the addition of 0.1% sodium laroylsarcosine and 1 mM dithiothreitol) in a ratio of 1:2 adding protease inhibitor cocktail (Sigma P2714). Denatured SDS PAGE followed by immunoblotting was used to detect the conjugates. Subsequently, the membrane was subjected to detection with Clarity™ Western ECL substrate (Biorad) and visualized with the ChemiDoc™ MP Imaging system (Biorad) according to the manufacturer's guidelines (FIG. 32).

Preliminary results indicated that approximately 33, 13 and 21 mg of rCHIR-AB1 proteins were produced per kilogram tobacco leaves using AGL-1, GV3101 pM90 or LBA4404 as Agrobacterium strains, respectively. The latter refer to protein being purified by immobilized metal ion affinity chromatography (IMAC). However, the recombinant protein poorly binds to IMAC and >95% of the protein flows through. Therefore, an estimated >600 mg of rCHIR-AB1-HRP protein is produced per Kg of N. bethamiana plant leaves with AGL-1 Agrobacterium-mediated infiltration of the expression cassette. Initial ELISA indicated that HRP genetically fused to rCHIR-AB1 is active in abundance in the crude extract (95%) but also in the IMAC purified extract to a lesser extend (5%).

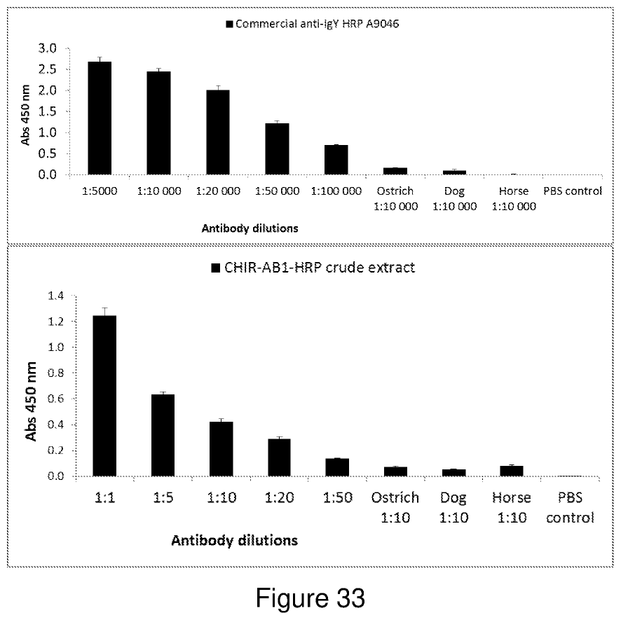

Subsequently, the rCHIR-AB1-HRP conjugate protein was purified from leaf tissue in either MES buffer (50 mM MES, 20 mM NaCl, pH 6.0) or a phosphate buffer (18 mM KH₂PO₄, 100 mM Na₂HPO₄, 27 mM KCL and 140 mM NaCl, pH 7.4), both with the addition of 0.04% sodium metabisulfite (Na₂S₂O₅). Using ELISA, it was confirmed once more that the HRP genetically fused to rCHIR-AB1 is active but also that the rCHIR-AB1 binds to the Fc region of chicken IgY and to a lesser extent to ostrich IgY (FIG. 33). The plant produced rCHIR-AB1-HRP conjugate protein can therefore be used as conjugate in ELISA detection of chicken IgY.

Enzyme-Linked Immunosorbent Assay (ELISA)

Fifty microliter (50 μl) volumes of an antibody dilution of 1:500 in PBS (chicken, ostrich, dog and horse) were coated into the wells of 96-well Maxisorp Nunc-Immunoplates, with overnight incubation at 4° C. Primary chicken and ostrich IgY were purified from eggs whilst serum antibodies for dogs and horses were used. Plates were washed 3× with 200 μl of PBS-T buffer. Fifty microliters per well of blocking buffer (4% Oxoid casein hydrolysate—acid in PBS buffer) was incubated for two hours at room temperature before being washed as before. Serial dilutions of the secondary antibodies of the Commercial anti-IgY HRP A9046 (Sigma Aldrich) (1:5000-1:100000) or plant produced rCHIR-AB1-HRP crude extract (1:1-1:50) were prepared in blocking buffer, and added in 50 μl volumes into wells. After incubation for two hours at room temperature, the plates were washed as before. After a final one hour incubation and wash step, 50 μl of 3,3′,5,5′-Tetramethylbenzidine (TMB) substrate (Sigma) was added to each well. Plates were incubated at room temperature for 10 minutes before the reaction was stopped with the addition of an equal volume of 2 M H₂SO₄. Absorbance was read at 450 nm on a HiDEX sense microplate reader.

### REFERENCES

Abolnik C, Fehrsen J, Olivier A, Van Wyngaardt W, Fosgate G & Ellis C, 2013. Serological investigation of highly pathogenic avian influenza H5N2 in ostriches (Struthio camelus) Avian Pathol. 2013; 42(3): 206-14.

Abolnik C, Pieterse R, Peyrot BM, Choma P, Phiri TP, Ebersohn K, van Heerden CJ, Vorster AA, van der Zel G, Geertsma PJ, Laleye AT, Govindasamy K & Rauff DL, 2018. The incursion and spread of HPAI H5N8 Clade 2.3.4.4 within South Africa. Avian Diseases. In press.

He Y and Bjorkman PJ, 2011. Structure of FcRY, an avian immunoglobulin receptor related to mammalian mannose receptors, and its complex with IgY. PNAS 108 (30): 12431-12436.

Moore, C., G. S. Cumming, J. Slingsby, and J. Grewar. Tracking socioeconomic vulnerability using network analysis: insights from an avian influenza outbreak in an ostrich production network. PLOS One 9(1): e86973. 2014.

RahimiRad, S., Alizadeh, A., Alizadeh, E. and Hosseini, S.M. The avian influenza H9N2 at avian-human interface: A possible risk for the future pandemics. J Res Med Sci. 2016 Jul 29;21:51. doi: 10.4103/1735-1995.187253. eCollection 2016. Review.

Rauff D, Strydom C, Abolnik C 2016. Evolutionary consequences of a decade of vaccination against subtype H6N2 influenza. Virology. Nov;498:226-239. doi: 10.1016/j.viro1.2016.08.035.

Sainsbury F, Thuenemann EC and Lomonossoff GP, 2009. pEAQ: versatile expression vectors for easy and quick transient expression of heterologous proteins in plants. Plant Biotechnology Journal 7: 682-693.

Sainsbury F, Caňizares MC and Lomonossoff GP, 2010. Cowpea mosaic virus: The plant virus-based biotechnology workhorse. Annual Review of Phytopathology 48:437-455.

Sainsbury F, Saxena P, Geisler K, Osbourn A and Lomonossoff GP, 2012. Using a virus-derived system to manipulate plant natural product biosynthetic pathways. In: Methods in Enzymology 517:185-202, Chapter 9. ISSN 0076-6879.

Shevchenko A, Tomas H, Havliš J, Olsen VJ and Mann M, 2007 In-gel digestion for mass spectrometric characterization of proteins and proteomes. Nature Protocols. 1, 2856-2860.

Strasser R, Stadlmann J, Schãhs M, Stiegler G, Quendler H, Mach L, Glőssi J, Weterings K, Pabst M and Steinkelner H, 2008. Generation of glycol-engineered Nicotiana benthamiana for the production of monoclonal antibodies with a homogenous human-like N-glycan structure. 6: 392-402.

Viertlboeck BC, Schweinsberg S, Hanczaruk MA, Schmitt R, Du Pasquier L, Herberg FW and Gabel TW, 2007. The chicken leukocyte receptor complex encodes a primordial, activating, high-affinity IgY Fc receptor. PNAS 104 (28): 11718-11723.

Webster, R. G., W. G. Bean, O. T. Gorman, T. M. Chambers, and Y. Kawaoka. Evolution and ecology of influenza A viruses. Microbiol. Rev. 56:152-179. 1992.

1. A method of detecting the presence of an antibody to an avian influenza hemagglutinin antigen in a sample from a subject, wherein the antibody binds to an epitope of the avian influenza hemagglutinin antigen, the method comprising the steps of:
i. cloning a codon optimised nucleic acid encoding a truncated H5, H6, H7 or H9 antigen into a vector;
ii. infiltrating a plant cell with the vector so as to express the truncated H5, H6, H7 or H9 antigen in the plant;
iii. recovering the truncated H5, H6, H7 or H9 antigen expressed by the plant;
iv. contacting the sample with at least one of the truncated H5, H6, H7 or H9 antigens, and
v. detecting the formation of an antibody-antigen complex, wherein the antibody-antigen complex comprises antibodies in the sample bound to one or more of the truncated H5, H6, H7 or H9 antigens;
wherein the formation of an antibody-antigen complex confirms that the subject has been exposed to an avian influenza hemagglutinin antigen.

2. The method of claim 1, wherein the truncated H5, H6, H7 or H9 antigens comprise a sequence of SEQ ID NO:3, 5, 7 or 9, respectively.

3. The method of claim 1, wherein the formation of the antibody-antigen complex is detected by either (i) the binding of a labelled secondary antibody to the antibody-antigen complex, or (ii) by the binding of a labelled secondary antigen to the antibody-antigen complex.

4. The method of claim 3, wherein the labelled secondary antibody is either conjugated to or is a genetic fusion with an indicator molecule.

5. The method of claim 4, wherein the labelled secondary antibody comprises the sequence of SEQ ID NO:15.

6. The method of claim 3, wherein the labelled secondary antigen is a truncated H5, H6, H7 or H9 antigen which is either conjugated to or is a genetic fusion with an indicator molecule.

7. The method of claim 4, wherein the indicator molecule is selected from horseradish peroxidase or alkaline phosphatase.

8. The method of claim 1, wherein the subject has been exposed to avian influenza.

9. The method of claim 1, wherein the sample is selected from the group consisting of blood, serum, plasma, saliva, conjunctival fluid urine and feces.

10. The method of claim 1, wherein the at least one truncated H5, H6, H7 or H9 antigen includes an affinity tag, and wherein the affinity tag facilitates the purification of the antigen.

11. The method of claim 10, wherein the affinity tag is a 6×-His tag.

12. The method of claim 1, wherein the at least one truncated H5, H6, H7 or H9 antigen is attached to or immobilized to a solid support.

13. The method of claim 12, wherein the solid support is selected from the group consisting of a bead, a flow path in a lateral flow immunoassay device, a well in a microtiter plate, or a flow path in a rotor.

14. The method of claim 1, wherein the formation of the antibody-antigen complex is detected by one or more of the following:
dip stick immunotesting, ELISA, flow cytometry, fluorescence, immunochip assay, immunochromatographic assay, immunoblot, latex agglutination, lateral flow assay, polarization, radioimmunoassay, and bead-based technology.

15. A device for assaying for the presence of an antibody to avian influenza hemagglutinin antigen in a sample from a subject, comprising:
(i) at least one antigen comprising a sequence selected from SEQ ID NO:3, 5, 7 or 9; and
(ii) a means for detecting the formation of an antibody-antigen complex between an antibody in the sample and the at least one antigen;
wherein the means for detecting the formation of an antibody-antigen complex comprises:
(a) a labelled secondary antibody; or
(b) a labelled secondary antigen;
wherein binding of the labelled secondary antibody or labelled secondary antigen to the antibody-antigen complex confirms that the subject has been exposed to an avian influenza hemagglutinin antigen.

16. The device of claim 15, wherein the labelled secondary antibody is either conjugated to or is a genetic fusion with an indicator molecule.

17. The device of claim 16, wherein the labelled secondary antibody comprises the sequence of SEQ ID NO:15.

18. The device of claim 15, wherein the labelled secondary antigen is a truncated H5, H6, H7 or H9 antigen which is either conjugated to or is a genetic fusion with an indicator molecule.

19. The device of claim 16, wherein the indicator molecule is selected from horseradish peroxidase or alkaline phosphatase.

20. The device of claim 15, wherein the subject has been exposed to avian influenza.

21. The device of claim 15, wherein the sample is selected from the group consisting of blood, serum, plasma, saliva and urine.

22. The device of claim 15, wherein the at least one antigen is attached to or immobilized to a solid support.

23. The device of claim 22, wherein the solid support is selected from the group consisting of a bead, a flow path in a lateral flow immunoassay device, a well in a microtiter plate, or a flow path in a rotor.

24. The device of claim 15, wherein the formation of the antibody-antigen complex is detected by one or more of the following: dip stick immunotesting, ELISA, flow cytometry, fluorescence, immunochip assay, immunochromatographic assay, immunoblot, latex agglutination, lateral flow assay, polarization, radioimmunoassay, and bead-based technology.
